(19) United States
(12) Patent Application Publication
(10) Pub. No.: US 2023/0416252 A1
(43) Pub. Date: Dec. 28, 2023

(54) PROCESS TOWARD THE MANUFACTURE OF (6R,10S)-10-{4-[5-CHLORO-2-(4-CHLORO-1H-1,2,3-TRIAZOL-1-YL)PHENYL]-6-OXO-1(6H)-PYRIMIDINYL}- 1-(DIFLUOROMETHYL)-6-METHYL-1,4,7,8,9,10-HEXAHYDRO-11,15-(METHENO)PYRAZOLO[4,3-B][1,7]DIAZACYCLOTETRADECIN-5(6H)-ONE

(71) Applicants: Bristol-Myers Squibb Company, Princeton, NJ (US); Janssen Pharmaceutica NV, Beerse (BE)

(72) Inventors: Subha MUKHERJEE, Princeton, NJ (US); William P. GALLAGHER, Princeton, NJ (US); Christopher Robert JAMISON, Princeton, NJ (US); Carolyn S. WEI, Belle Mead, NJ (US); Sergei KOLOTUCHIN, Princeton, NJ (US); Amarjit SINGH, Highland Park, NJ (US); Nicolas CUNIERE, Princeton, NJ (US); Chris SFOUGGATAKIS, Princeton, NJ (US); Adrian ORTIZ, Oak Park, CA (US); Steven R. WISNIEWSKI, Princeton, NJ (US); Bin ZHENG, Princeton, NJ (US); Helen Y. LUO, San Francisco, CA (US); Sébastien François Emmanuel LEMAIRE, Beerse (BE); Cyril BEN HAÏM, Beerse (BE); Kostiantyn CHERNICHENKO, Beerse (BE); Diego Fernando Domenico BROGGINI, Schaffhausen (CH); Simon Albert WAGSCHAL, Schaffhausen (CH); Duy Chi Trung CAO, Schaffhausen (CH); Karl REUTER, Gundelfingen (DE); Björn SCHMALZBAUER, Waldkirch (DE); Philipp KOSCHKER, Freiburg (DE); Martin D. EASTGATE, Princeton, NJ (US); Dimitri SKLIAR, Princeton, NJ (US); Alexander ZHDANKO, Beerse (BE); Christos XIOURAS, Beerse (BE); Matthew Penfield MOWER, Beerse (BE); Ngoc Duc TRAN, Beerse (BE); Ramdane RAHMANI, Beerse (BE); Xavier Jean-Marie JUSSEAU, Beerse (BE); Kiran MATCHA, Beerse (BE); Luca Alessandro PEREGO, Schaffhausen (CH)

(21) Appl. No.: 18/031,277

(22) PCT Filed: Oct. 11, 2021
(86) PCT No.: PCT/US2021/054413

§ 371 Date: Apr. 11, 2023

Related U.S. Application Data
(60) Provisional application No. 63/090,558, filed on Oct. 12, 2020.

(51) Int. Cl. C07D 471/16; B01J 23/72

(52) U.S. Cl. CPC C07D 471/16; B01J 23/72; C07B 2200/13

(57) ABSTRACT

The present application generally relates to several processes for the preparation of (6R,10S)-10-{4-[5-chloro-2-(4-chloro-1H-1,2,3-triazol-1-yl)phenyl]-6-oxo-1(6H)-pyrimidinyl}-1-(difluoromethyl)-6-methyl-1,4,7,8,9,10-hexahydro-11,15-(metheno)pyrazolo[4,3-b][1,7]diazacyclotetradecin-5(6H)-one: Compound (I).

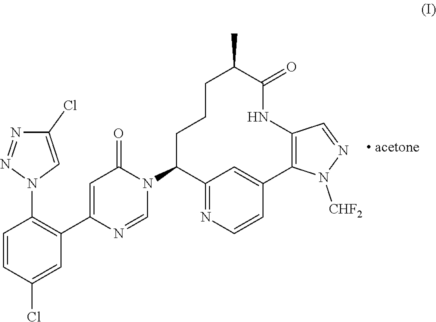 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

### TECHNICAL FIELD

The present disclosure generally relates to several processes for the preparation of (6R,10S)-10-{4-[5-chloro-2-(4-chloro-1H-1,2,3-triazol-1-yl)phenyl]-6-oxo-1(6H)-pyrimidinyl}-1-(difluoromethyl)-6-methyl-1,4,7,8,9,10-hexahydro-11,15-(metheno)pyrazolo[4,3-b][1,7]diazacyclotetradecin-5(6H)-one, a FXIa inhibitor useful for the treatment of thromboembolic disorders, which include venous thrombosis and deep vein thrombosis.

### BACKGROUND

Factor XIa is a plasma serine protease involved in the regulation of blood coagulation, which is initiated in vivo by the binding of tissue factor (TF) to factor VII (FVII) to generate factor VIIa (FVIIa). The resulting TF:FVIIa complex activates factor IX (FIX) and factor X (FX) that leads to the production of factor Xa (FXa). The generated FXa catalyzes the transformation of prothrombin into small amounts of thrombin before this pathway is shut down by tissue factor pathway inhibitor (TFPI). The process of coagulation is then further propagated via the feedback activation of Factors V, VIII and XI by catalytic amounts of thrombin. (Gailani, D. et al., Arterioscler. Thromb. Vasc. Biol., 27:2507-2513 (2007).) The resulting burst of thrombin converts fibrinogen to fibrin that polymerizes to form the structural framework of a blood clot, and activates platelets, which are a key cellular component of coagulation (Hoffman, M., Blood Reviews, 17:S1-S5 (2003)). Therefore, factor XIa plays a key role in propagating this amplification loop and is thus an attractive target for anti-thrombotic therapy.

U.S. Pat. No. 9,453,018 discloses macrocycle compounds as factor XIa inhibitors useful for the treatment of thromboembolic disorders. One of the compounds has the following structure:

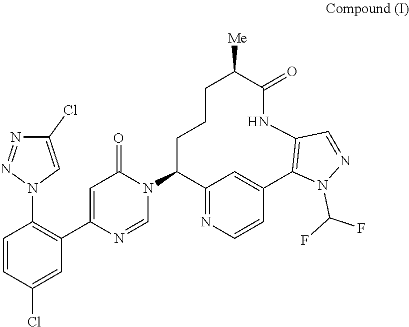 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

However, there are difficulties associated with the adaptation of the multistep synthesis disclosed in U.S. Pat. No. 9,453,018 to a larger scale synthesis, such as production in a pilot plant or on a manufacturing scale. One difficulty is that the Grubbs (II) reagent was not readily adaptable to commercial scale synthesis due to its high costs. Further, there is a continuing need to find a process that provides higher yields in order to improve manufacturing economics and/or reduce waste. Preferably, a new process will employ less expensive starting materials.

The present application is directed to processes that are suitable for preparing larger quantities of Compound (I) than is typically prepared by laboratory scale processes. The present application is also directed to processes that provides higher yields of Compound (I) than the previously disclosed processes on a manufacturing scale.

Specifically, alternative novel compounds are employed to achieve Compound (I), which both shorten the amount of synthetic steps, are less expensive, and ultimately afford higher yields.

### SUMMARY

Described herein are several processes toward the preparation of (6R,10S)-10-{4-[5-chloro-2-(4-chloro-1H-1,2,3-triazol-1-yl)phenyl]-6-oxo-1(6H)-pyrimidinyl}-1-(difluoromethyl)-6-methyl-1,4,7,8,9,10-hexahydro-11,15-(metheno)pyrazolo[4,3-b][1,7]diazacyclotetradecin-5(6H)-one.

Accordingly, in a first aspect, the invention provides a process for preparing a crystalline solvate form of a compound represented by:

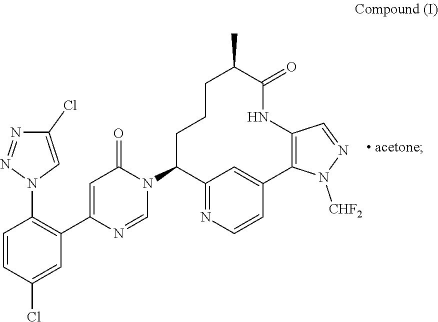 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

comprising the steps of:
(a) reacting Compound A having the structure:

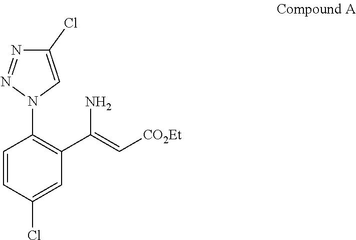 [SMILES: CCOC(=O)/C=C(\N)c1cc(Cl)ccc1-n1cc(Cl)nn1]

with N,N-dimethylformamide dimethyl acetal in a suitable solvent to yield a mixture containing methanol as a by-product;
(b) to the mixture of step (a) adding Compound C having the structure:

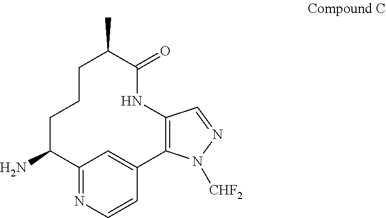 [SMILES: C[C@@H]1CCC[C@H](N)c2cc(ccn2)-c2c(cnn2C(F)F)NC1=O]

to yield the crystalline solvate form of Compound (I):

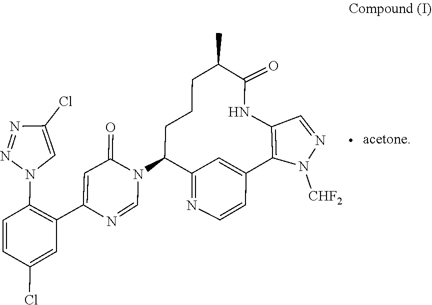 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

In an embodiment of the process for preparing the acetone solvate form of Compound (I), methanol is removed before step (b).

In an embodiment of the process for preparing the acetone solvate form of Compound (I), acetic acid is added after the removal of methanol from the mixture of step (a).

In another embodiment of the process for preparing the acetone solvate form of Compound (I), trimethylamine is added after the addition of Compound C in step (b).

In an embodiment of the process for preparing the acetone solvate form of Compound (I), Compound (I) is crystallized in a mixture of methanol and water followed by a rinse with aqueous acetone to yield the crystalline acetone solvate form of Compound (I).

In a second aspect, the invention provides a crystalline solvate form of:

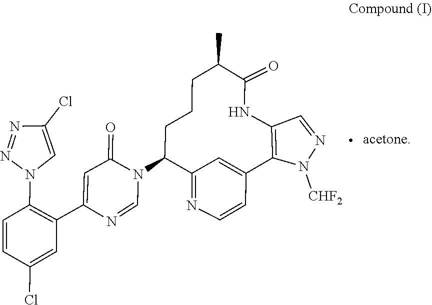 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

In an embodiment of the X-ray powder diffraction pattern of the crystalline solvate form of Compound (I), the X-ray powder diffraction pattern comprises one, two, three or four peaks selected peaks expressed in values of degrees 2Θ at 20.0±0.2, 21.3±0.2, 21.6±0.2, and 23.9±0.2.

In an embodiment of the crystalline acetone solvate form of Compound (I), the crystalline form exhibits a Fourier transform infrared spectrum having characteristic peaks expressed in units of reciprocal wave numbers (cm⁻¹) at values of about 1709, about 1676, about 1532, about 1485, about 1457, about 1441, about 1432, about 1370, about 1291, about 1219, about 1189, about 1135, about 1119, about 1068, about 1039, about 994, about 942, about 883, about 827, about 801, and about 696.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 illustrates the XRPD spectrum of the crystalline form of Compound (I) as the acetone solvate.

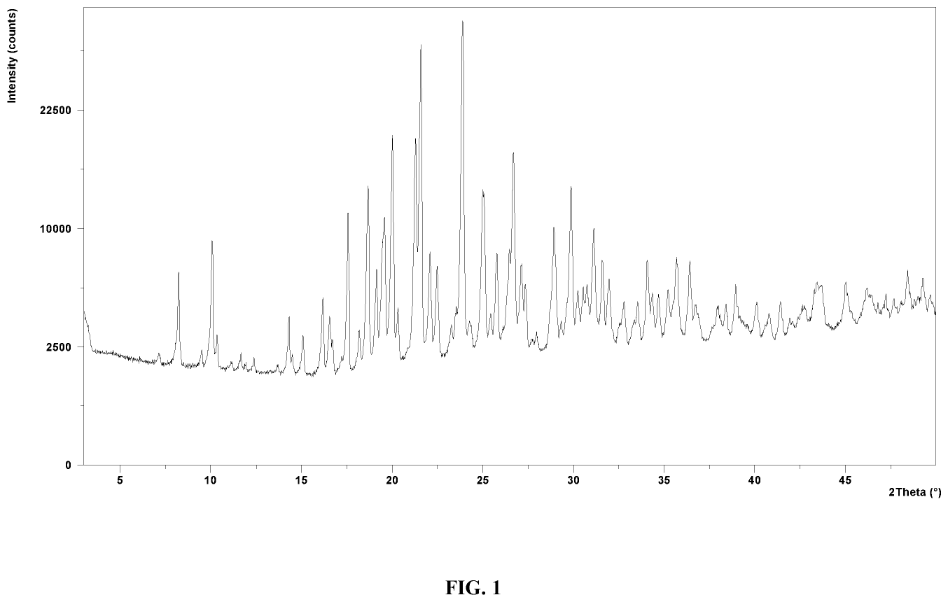

FIG. 2 illustrates the IR spectrum of the crystalline form of Compound (I) as the acetone solvate.

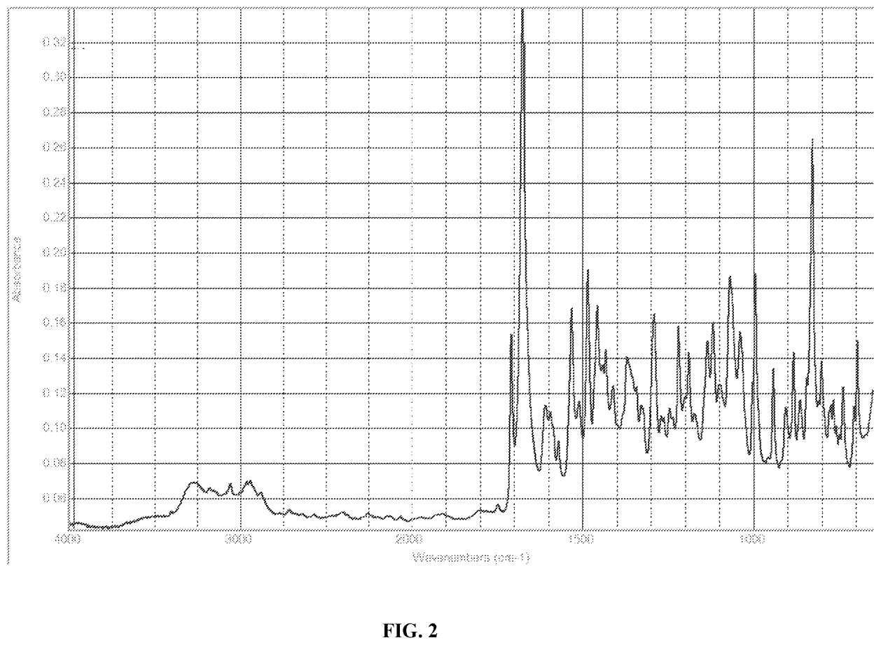

### DETAILED DESCRIPTION

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise.

The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

### Definitions

As used herein, “pharmaceutically acceptable salts” refer to derivatives of the disclosed compounds wherein the parent compound is modified by making acid or base salts thereof. Both the free form and the salts of these end products are within the scope of the invention.

Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans-(or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

IR spectroscopy, particularly FT-IR, is a technique that may be used to characterize solid forms together with or separately from X-ray powder diffraction. In an IR spectrum, absorbed light is plotted on the x-axis of a graph in the units of “wavenumber” (cm⁻¹), with intensity on the y-axis. Variation in the position of IR peaks also exists and may be due to sample conditions as well as data collection and processing. The typical variability in IR spectra reported herein is on the order of plus or minus 2.0 cm⁻¹. Thus, the use of the word “about” when referencing IR peaks is meant to include this variability and all IR peaks disclosed herein are intended to be reported with such variability.

The term “reducing agent” refers to any reagent that will decrease the oxidation state of a carbon atom in the starting material by either adding a hydrogen atom to this carbon or adding an electron to this carbon and as such would be obvious to one of ordinary skill and knowledge in the art. The definition of “reducing reagent” includes but is not limited to: borane-dimethyl sulfide complex, 9-borabicyclo[3.3.1]nonane (9-BBN), catechol borane, lithium borohydride, sodium borohydride, sodium borohydride-methanol complex, potassium borohydride, sodium hydroxyborohydride, lithium triethylborohydride, lithium n-butylborohydride, sodium cyanoborohydride, calcium (II) borohydride, lithium aluminum hydride, diisobutylaluminum hydride, n-butyl-diisobutylaluminum hydride, sodium bis-methoxyethoxyaluminum hydride, triethoxysilane, diethoxymethylsilane, lithium hydride, lithium, sodium, hydrogen Ni/B, and the like. Certain acidic and Lewis acidic reagents enhance the activity of reducing reagents. Examples of such acidic reagents include: acetic acid, methanesulfonic acid, hydrochloric acid, and the like. Examples of such Lewis acidic reagents include: trimethoxyborane, triethoxyborane, aluminum trichloride, lithium chloride, vanadium trichloride, dicyclopentadienyl titanium dichloride, cesium fluoride, potassium fluoride, zinc (II) chloride, zinc (II) bromide, zinc (II) iodide, and the like.

The term “oxidizing agent” refers to a chemical species that undergoes a chemical reaction in which it gains one or more electrons. An oxidizing agent also refers to any substance which increases the number of bonds to oxygen or decreases the number of bonds to hydrogen. Examples of oxidizing agents include, but are not limited to: oxygen (O₂), ozone (O₃), hydrogen peroxide (H₂O₂) and other inorganic peroxides, Fenton's reagent, fluorine (F₂), chlorine (Cl₂), and other halogens, nitric acid (HNO₃) and nitrate compounds, sulfuric acid (H₂SO₄), peroxydisulfuric acid (H₂S₂O₈), peroxymonosulfuric acid (H₂SO₅), hypochlorite, chlorite, chlorate, perchlorate, and other analogous halogen compounds like household bleach (NaClO), hexavalent chromium compounds such as chromic and dichromic acids and chromium trioxide, pyridinium chlorochromate (PCC), and chromate/dichromate compounds, permanganate compounds such as potassium permanganate (KMnO₄), sodium perborate (NaBO₃), nitrous oxide (N₂O), nitrogen dioxide/dinitrogen tetroxide (NO₂/N₂O₄), potassium nitrate (KNO₃), the oxidizer in black powder sodium bismuthate (NaBiO₃), cerium (IV) compounds such as ceric ammonium nitrate and ceric sulfate, lead dioxide (PbO₂).

The term “dehydrating agent” refers to any substance that dries or removes water from a material. In chemical reactions where dehydration occurs, the reacting molecule loses a molecule of water. A dehydrating agent can also refer to any substance that drives a dehydration reaction. Examples include, but are not limited to: phosphoroxychloride (POCl₃), (COCl)₂, PCl₅, SOCl₂, PCl₃, dimethylchloroformiminium chloride (ClCH═N(CH₃)₂Cl, N,N′-dicyclohexylcarbodiimide (DCC), 1-ethyl-3-(3-dimethylaminopropyl)carbodiimide (EDC), N,N′-diisopropylcarbodiimide (DIC), 1-cyclohexyl-(2-morpholinoethyl)carbodiimide metho-p-toluene sulfonate (CMCT), methanesulfonyl chloride (MsCl) or 4-toluenesulfonyl chloride (TsCl) with organic bases, TiCl₄ with organic bases, PPh₃ with CCl₄ or CBr₄, SO₃·Py, pivaloyl chloride with organic bases, and cyanuric chloride with organic bases.

The term “removable protecting group” or “protecting group” refers to any group which when bound to a functionality, such as the oxygen atom of a hydroxyl or carboxyl group or the nitrogen atom of an amine group, prevents reactions from occurring at these functional groups and which protecting group can be removed by conventional chemical or enzymatic steps to reestablish the functional group. The particular removable protecting group employed is not critical.

The term “ligand” as used herein refers to a phosphine derivative that ligates palladium such as a mono or bi-dentate aryl or alkyl phosphine, which is capable of complexing a palladium atom. The term is well known to one skilled in the particular art.

The term “chlorinating agent” or “chlorination” refers to any substance which adds a chlorine atom to a substrate. A chlorinating agent is a specific type of oxidizing agent. Examples include, but are not limited to: 1,3-dichloro-5,5-dimethylhydantoin, NCS, NaClO, trichloroisocyanuric acid, thionyl chloride (SOCl₂), oxalyl chloride ((COCl)₂).

Abbreviations as used herein, are defined as follows:
Me methyl
Et ethyl
Pr propyl
i-Pr isopropyl
Bu butyl
i-Bu isobutyl
t-Bu tert-butyl
Ph phenyl
Bn benzyl
Boc or BOC tert-butyloxycarbonyl
Boc₂O di-tert-butyl dicarbonate
AcOH or HOAc acetic acid
AlCl₃ aluminum chloride
AIBN azobisisobutyronitrile
aqueous aq
BBr₃ boron tribromide
BCl₃ boron trichloride
BEMP 2-tert-butylimino-2-diethylamino-1,3-dimethylperhydro-1,3,2-diazaphosphorine
BOP reagent benzotriazol-1-yloxytris(dimethylamino)phosphonium hexafluorophosphate
Burgess reagent 1-methoxy-N-triethylammoniosulfonyl-methanimidate
Cbz carbobenzyloxy
DCM or CH₂Cl₂ dichloromethane
CH₃CN or ACN acetonitrile
CDCl₃ deutero-chloroform
CHCl₃ chloroform
mCPBA or m-CPBA meta-chloroperbenzoic acid
Cs₂CO₃ cesium carbonate
Cu(OAc)₂ copper (II) acetate
CuI copper(I) iodide
CuSO₄ copper(II) sulfate
Cy₂NMe N-cyclohexyl-N-methylcyclohexanamine
DBU 1,8-diazabicyclo[5.4.0]undec-7-ene
DCE 1,2-dichloroethane
DEA diethylamine
Dess-Martin 1,1,1-tris(acetyloxy)-1,1-dihydro-1,2-beniziodoxol-3-(1H)-one
DIC or DIPCDI diisopropylcarbodiimide
DIEA, DIPEA or Hunig's diisopropylethylamine base
DMAP 4-dimethylaminopyridine
DME 1,2-dimethoxyethane
DMF dimethyl formamide
DMSO dimethyl sulfoxide
cDNA complimentary DNA
Dppp (R)-(+)-1,2-bis(diphenylphosphino)propane
DuPhos (+)-1,2-bis((2S,5S)-2,5-diethylphospholano)benzene
EDC N-(3-dimethylaminopropyl)-N-ethylcarbodiimide
EDCI N-(3-dimethylaminopropyl)-N-ethylcarbodiimide hydrochloride
EDTA ethylenediaminetetraacetic acid
(S,S)-EtDuPhosRh(I) (+)-1,2-bis((2S,5S)-2,5-diethylphospholano)benzene(1,5-cyclooctadiene)rhodium(I) trifluoromethanesulfonate
Et₃N or TEA triethylamine
EtOAc ethyl acetate
Et₂O diethyl ether
EtOH ethanol
GMF glass microfiber filter
Grubbs II (1,3-bis(2,4,6-trimethylphenyl)-2-imidazolidinylidene)dichloro (phenylmethylene)(triycyclohexylphosphine)ruthenium
HCl hydrochloric acid
HATU O-(7-azabenzotriazol-1-yl)-N,N,N′,N′-tetramethyluronium hexafluorophosphate
HEPES 4-(2-hydroxyethyl)piperazine-1-ethanesulfonic acid
Hex hexane
HOBt or HOBT 1-hydroxybenzotriazole
H₂O₂ hydrogen peroxide
H₂SO₄ sulfuric acid
IBX 2-iodoxybenzoic acid
InCl₃ Indium(III) chloride
Jones reagent CrO₃ in aqueous H₂SO₄, 2 M
K₂CO₃ potassium carbonate
K₂HPO₄ potassium phosphate dibasic
K₃PO₄ potassium phosphate tribasic
KOAc potassium acetate
K₃PO₄ potassium phosphate
LAH lithium aluminum hydride
LG leaving group
LiOH lithium hydroxide
MeOH methanol
MgSO₄ magnesium sulfate
MsOH or MSA methylsulfonic acid
NaCl sodium chloride
NaH sodium hydride
NaHCO₃ sodium bicarbonate
Na₂CO₃ sodium carbonate
NaOH sodium hydroxide
Na₂SO₃ sodium sulfite
Na₂SO₄ sodium sulfate
NBS N-bromosuccinimide
NCS N-chlorosuccinimide
NH₃ ammonia
NH₄Cl ammonium chloride
NH₄OH ammonium hydroxide
NH₄COOH ammonium formate
NMM N-methylmorpholine
OTf triflate or trifluoromethanesulfonate
Pd(allyl)(CatacxiumA)Cl Palladium allyl chloride Di(1-adamantyl)-n-butylphosphine
Pd₂(dba)₃ tris(dibenzylideneacetone)dipalladium(O)
Pd(OAc)₂ palladium(II) acetate
Pd/C palladium on carbon
Pd(dppf)Cl₂ [1,1′-bis(diphenylphosphino)-ferrocene]dichloropalladium(II)
Ph₃PCl₂ triphenylphosphine dichloride
PG protecting group
POCl₃ phosphorus oxychloride
PMDTA N,N,N′,N″,N″-pentamethyldiethylenetriamine
i-PrOH or IPA isopropanol
PS Polystyrene
rt room temperature
SEM-Cl 2-(trimethysilyl)ethoxymethyl chloride
SiO₂ silica oxide
SnCl₂ tin(II) chloride
TBAI tetra-n-butylammonium iodide
TBN t-butyl nitrite
TFA trifluoroacetic acid
THF tetrahydrofuran
TMEDA tetramethylethylenediamine
TMSCHN₂ trimethylsilyldiazomethane
T3P® propane phosphonic acid anhydride
TRIS tris (hydroxymethyl) aminomethane
pTsOH p-toluenesulfonic acid

Additional abbreviations as used herein are as follows: “° C.” for degrees Celsius, “eq” or “equiv.” for equivalent or equivalents, “aq.” for aqueous, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “RBF” for round bottom flask, “atm” for atmosphere, “psi” for pounds per square inch, “conc.” for concentrate, “RCM” for ring-closing metathesis, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “nOe” for nuclear Overhauser effect spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

### Embodiments

The present application is directed to a number of synthetic intermediates and processes for preparing those intermediates and Compound (I).

General aspects of these exemplary methods are described in the schemes and the Examples. Each of the products of the following processes is optionally separated, isolated, and/or purified prior to its use in subsequent processes.

Generally, the reaction conditions such as temperature, reaction time, solvents, work-up procedures, and the like, will be those common in the art for the particular reaction to be performed. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

In one embodiment, the present invention provides a process for the preparation of a Compound (I). In an embodiment, the preparation of Compound (I)·acetone:

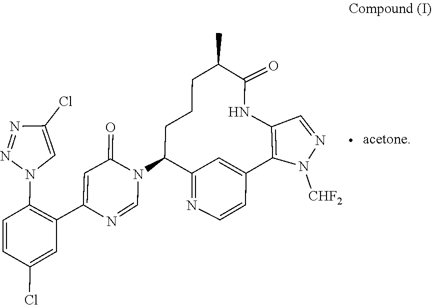 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

comprises the steps of:
(a) reacting Compound A having the structure:

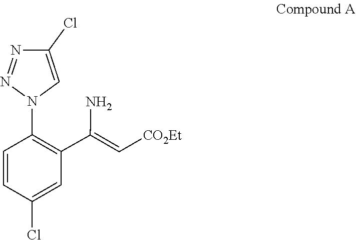 [SMILES: CCOC(=O)/C=C(\N)c1cc(Cl)ccc1-n1cc(Cl)nn1]

with N,N-dimethylformamide dimethyl acetal in a suitable solvent to yield a mixture containing methanol as a by-product;
(b) to the mixture of step (a) adding Compound C having the structure:

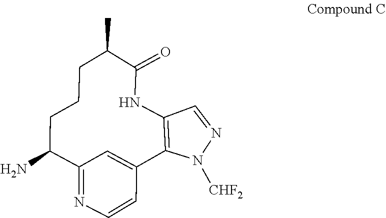 [SMILES: C[C@@H]1CCC[C@H](N)c2cc(ccn2)-c2c(cnn2C(F)F)NC1=O]

to yield the crystalline solvate form of Compound (I):

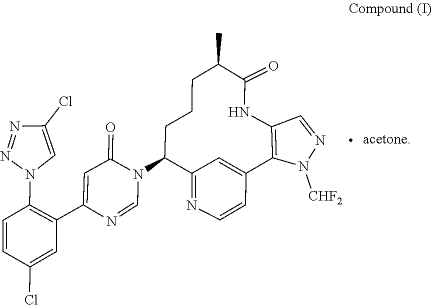 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

In an embodiment of the process for preparing the acetone solvate form of Compound (I), methanol is removed before step (b).

In an embodiment of the process for preparing the acetone solvate form of Compound (I), acetic acid is added after the removal of methanol from the mixture of step (a).

In another embodiment of the process for preparing the acetone solvate form of Compound (I), triethylamine is added after the addition of Compound C in step (b).

In an embodiment of the process for preparing the acetone solvate form of Compound (I), Compound (I) is crystallized in a mixture of acetone and water followed by a rinse with aqueous acetone to yield the crystalline acetone solvate form of Compound (I).

In an embodiment of the process for preparing the acetone solvate form of Compound (I), the suitable solvent for reaction of Compound A with N,N-dimethylformamide dimethyl acetal is selected from the group consisting of acetonitrile (ACN), dichloromethane (DCM), toluene, tetrahydrofuran (THF), tert-butyl methyl ether (MTBE), and ethyl acetate.

In a related aspect, the invention provides a process for preparing Compound A having the structure:

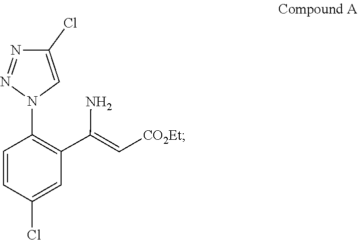 [SMILES: CCOC(=O)/C=C(\N)c1cc(Cl)ccc1-n1cc(Cl)nn1]

comprising the steps of:
(a) reacting Compound 1 having the structure:

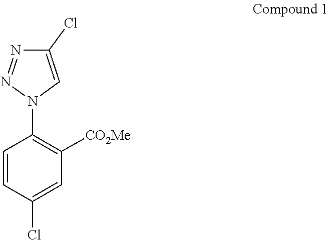 [SMILES: COC(=O)c1cc(Cl)ccc1-n1cc(Cl)nn1]

with ammonia (NH₃) in a suitable solvent to afford Compound 2 having the structure:

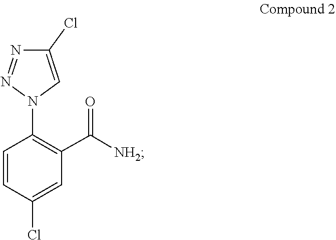 [SMILES: NC(=O)c1cc(Cl)ccc1-n1cc(Cl)nn1]

(b) reacting Compound 2 with a dehydrating agent to afford Compound 3 having the structure:

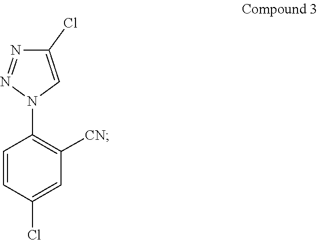 [SMILES: N#Cc1cc(Cl)ccc1-n1cc(Cl)nn1]

(c) reacting Compound 3 with potassium ethyl malonate, a base, and a Lewis Acid to afford Compound A:

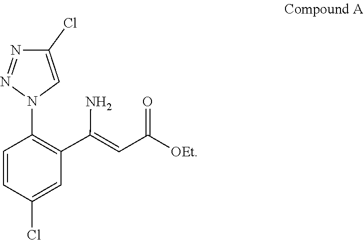 [SMILES: CCOC(=O)/C=C(\N)c1cc(Cl)ccc1-n1cc(Cl)nn1]

In an embodiment of the process for preparing Compound A, calcium chloride is added as a catalyst in step (a).

In another embodiment of the process for preparing Compound A, the dehydrating agent of step (b) is selected from the group consisting of phosphoroxychloride (POCl₃), (COCl)₂, PCl₅, SOCl₂ PCl₃, dimethylchloroformiminium chloride (ClCH═N(CH₃)₂Cl, N,N′-dicyclohexylcarbodiimide (DCC), 1-Ethyl-3-(3-dimethylaminopropyl)carbodiimide (EDC), N,N′-diisopropylcarbodiimide (DIC), 1-cyclohexyl-(2-morpholinoethyl)carbodiimide metho-p-toluene sulfonate (CMCT), methanesulfonyl chloride (MsCl) or 4-toluenesulfonyl chloride (TsCl) with organic bases, TiCl₄ with organic bases, PPh₃ with CCl₄ or CBr₄, SO₃·Py, pivaloyl chloride with organic bases, and cyanuric chloride with organic bases. In a further embodiment, the dehydrating agent of step (b) is selected from the group consisting of phosphoroxychloride (POCl₃), (COCl)₂, PCl₅, SOCl₂ PCl₃, and dimethylchloroformiminium chloride (ClCH═N(CH₃)₂Cl.

In an embodiment of the process for preparing Compound A, the Lewis Acid of step (c) is selected from the group consisting of zinc chloride (ZnCl₂), aluminum trichloride (AlCl₃), and boron trifluoride (BF₃).

In another embodiment of the process for preparing Compound A, the base of step (c) is selected from the group consisting of triethylamine, N,N-diisopropylethylamine (DIPEA), 1,8-diazabicyclo[5.4.0]undec-7-ene (DBU), 1,5-diazabicyclo[4.3.0]non-5-ene (DBN), 1,4-diazabicyclo[2.2.2]octane (DABCO), tetramethylethylenediamine (TMEDA), and N,N,N′,N″,N″-pentamethyldiethylenetriamine (PMDTA).

In an embodiment of the process for preparing Compound A, the suitable solvent for dissolving ammonia is selected from the group consisting of methanol, dioxane, ethanol, isopropanol, tetrahydrofuran (THF), and water.

In a related aspect, the invention provides a process for preparing Compound C having the structure:

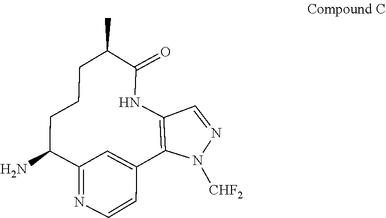 [SMILES: C[C@@H]1CCC[C@H](N)c2cc(ccn2)-c2c(cnn2C(F)F)NC1=O]

comprising the steps of:
(a) dissolving the hydrochloric acid salt of Compound 15 having the structure:

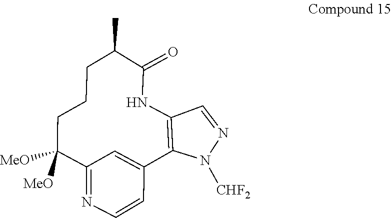

in a suitable solvent,
(b) then adding transaminase ATA-486 and an enzymatic catalyst to afford Compound C:

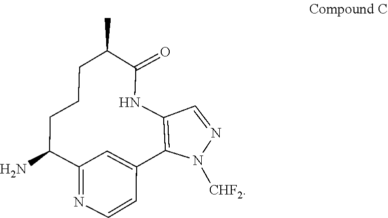 [SMILES: C[C@@H]1CCC[C@H](N)c2cc(ccn2)-c2c(cnn2C(F)F)NC1=O]

In an embodiment of the process for preparing Compound C, the enzymatic catalyst is pyridoxal 5′-phosphate hydrate (5-PLP).

In an aspect of the preparation of Compound C, the present application provides a process for preparing the hydrochloric acid salt of Compound 15 having the structure:

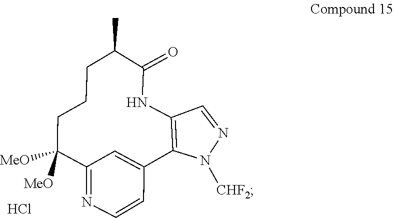

comprising the steps:
(a) reacting Compound 8 having the structure:

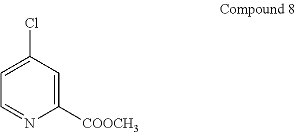 [SMILES: COC(=O)c1cc(Cl)ccn1]

with 2-methylcyclopentanone and a strong base to afford Compound 9 having the structure:

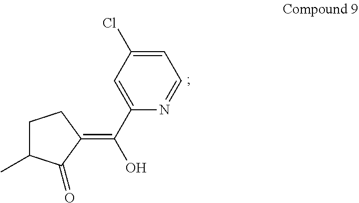 [SMILES: CC1CC/C(=C(/O)c2cc(Cl)ccn2)C1=O]

(b) reacting Compound 9 with an aqueous acid to afford Compound 10 having the structure:

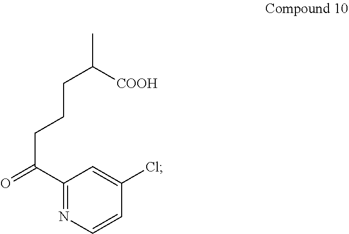 [SMILES: CC(CCCC(=O)c1cc(Cl)ccn1)C(=O)O]

(c) reacting Compound 10 with (1R,2S)-erythro-2-amino-1,2-diphenylethanol to afford the diastereomeric salt of Compound 10A:

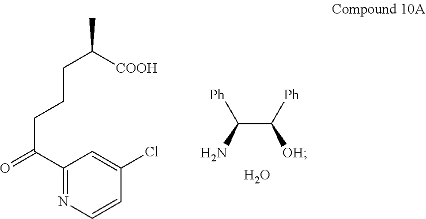

(d) dissolving the diastereomeric salt of Compound 10A with aqueous acid and a suitable organic solvent to afford Compound 10A:

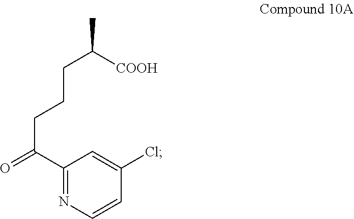 [SMILES: C[C@H](CCCC(=O)c1cc(Cl)ccn1)C(=O)O]

(e) reacting Compound 10A with trimethylsilyl chloride followed by trimethylorthoformate and then a strong base to afford Compound 11:

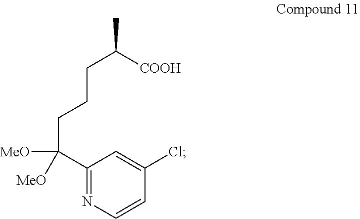 [SMILES: COC(CCC[C@@H](C)C(=O)O)(OC)c1cc(Cl)ccn1]

(f) mixing Compound 11 with dicyclohexylamine (DCHA) to afford the salt of Compound 11:

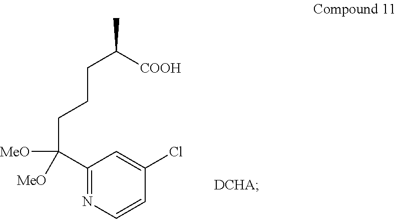 [SMILES: COC(CCC[C@@H](C)C(=O)O)(OC)c1cc(Cl)ccn1]

(g) reacting the dicyclohexylamine salt of Compound 11 with a coupling agent and the hydrochloric salt of

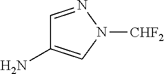

to form Compound 14 having the structure:

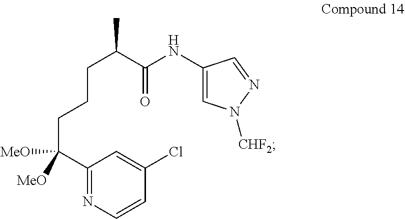

(h) Compound 14 was reacted with a metal catalyst followed by hydrochloric acid to afford the hydrochloric acid salt of Compound 15:

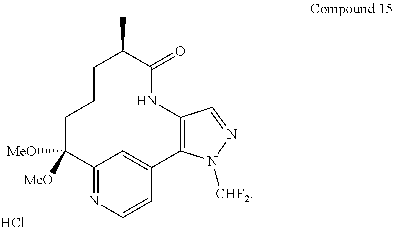

In an embodiment of the process for preparing Compound 15, the coupling agent of step (g) is selected from the group consisting of 1,1′-carbonyldiimidazole (CDI), dicyclohexyl carbodiimide (DCC), 1-ethyl-3-(3-dimethylaminopropyl)carbodiimide (EDCI) and 1,1′-thiocarbonyldiimidazole (TCDI).

In an embodiment of the process for preparing Compound 15, the aqueous acid for the step of preparing Compound 10 is selected from the group consisting of aqueous methane sulfonic acid, aqueous sulfuric acid, and aqueous hydrogen chloride.

In an embodiment of the process for preparing Compound 15, the suitable organic solvent for the step of preparing Compound 10A is selected from the group consisting of methanol, ethanol, and 2-butanone.

In an embodiment of the process for preparing Compound 15, the strong base for the step of preparing Compound 11 is selected from the group consisting of sodium hydroxide, lithium hydroxide, potassium hydroxide, and barium hydroxide.

In an embodiment, the present invention provides a process for obtaining the (R)-enantiomer of Compound 10, which comprises fractional crystallizing said (R)-enantiomer as its diastereomeric salt with a non-racemic 2-amino-1,2-diarylethanol from a solution or suspension of a mixture of the enantiomers of Compound 10 in a solvent, wherein the non-racemic 2-amino-1,2-diarylethanol is preferably non-racemic erythro-2-amino-1,2-diarylethanol and especially is (1R,2S)-2-amino-1,2-diarylethanol. Particular preference is given here to the fractional crystallization of (R)-Compound 10 as its diastereomeric salt with non-racemic 2-amino-1,2-diphenylethanol, preferably non-racemic erythro-2-amino-1,2-diphenylethanol and specifically (1R,2S)-2-amino-1,2-diphenylethanol.

Likewise, the invention also provides a process for obtaining the (S)-enantiomer of Compound 10, which comprises fractional crystallizing said (S)-enantiomer as its diastereomeric salt with non-racemic 2-amino-1,2-diarylethanol from a solution or suspension of a mixture of the enantiomers of Compound 10 in a solvent, wherein non-racemic 2-amino-1,2-diarylethanol is preferably non-racemic erythro-2-amino-1,2-diarylethanol, and especially is (1S,2R)-2-amino-1,2-diphenylethanol.

In an embodiment of the fractional crystallization process for obtaining the (R)-enantiomer of Compound 10, the solvent for the fractional crystallization process is selected from the group consisting of methanol, ethanol, and 2-butanone with or without water.

The present invention further relates to a process for the racemization of a non-racemic mixture of the enantiomers of Compound 10. Such a non-racemic mixture is contained for example in the mother liquor resulting from the aforementioned process for enantiomerically enriching Compound 10 by fractional crystallization of one of its enantiomers with non-racemic 2-amino-1,2-diarylethanol.

In the first step of this racemization process of the present invention Compound 10 is protected by converting its carboxylic acid group into an ester group. In a subsequent step the thus obtained protected derivative (Compound 41) is racemized at its chiral center by treatment with a strong base, such as potassium tert butoxide, potassium tert amylate, TBD (triazabicyclodecene) and MTBD (7-methyl-1,5,7-triazabicyclo(4.4.0)dec-5-ene to afford Compound 11. Suitable solvents for the racemization reaction are toluene, acetonitrile, MTBE, and methyl actetate. In a final step Compound 11 is deprotected so that a racemic mixture of the enantiomers of Compound 10 is obtained.

In an embodiment of the process for preparing Compound 15, the metal catalyst of step (h) is selected from the group consisting of palladium and ruthenium. In an embodiment of the process from preparing Compound 15, the metal catalyst of step (h), the metal catalyst comprises a palladium catalyst selected from the group consisting of Pd(OAc)₂/Cy₃P—HBF₄, [Pd(allyl) Cl]₂/Ad₂nBuP, and Pd(OAc)₂/BippyPhos. In an embodiment of the process for preparing Compound 15, the metal catalyst of step (h), the metal catalyst comprises a ruthenium catalyst selected from the group consisting of Ru₃(CO)₁₂, RuH₂(CO)(PPh₃)₃, and [RuCl₂(C₆H₆)]₂.

In an aspect, the invention provides a crystalline solvate form of:

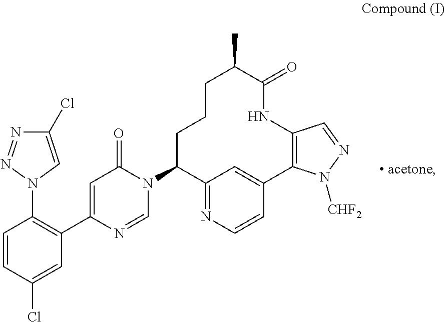 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

In an embodiment of the X-ray powder diffraction pattern of the crystalline solvate form of Compound (I), the X-ray powder diffraction pattern comprises one, two, three or four peaks selected from peaks expressed in values of degrees 2Θ at 20.0±0.2, 21.3±0.2, 21.6±0.2, and 23.9±0.2.

In an embodiment of the crystalline acetone solvate form of Compound (I), the crystalline form exhibits a Fourier transform infrared spectrum having characteristic peaks expressed in units of reciprocal wave numbers (cm⁻¹) at values of about 1709, about 1676, about 1532, about 1485, about 1457, about 1441, about 1432, about 1370, about 1291, about 1219, about 1189, about 1135, about 1119, about 1068, about 1039, about 994, about 942, about 883, about 827, about 801, and about 696.

In an embodiment of the invention, the present application provides an alternative process for the preparation of Compound C via Claisen rearrangement.

In an embodiment, the process for preparing Compound C having the structure:

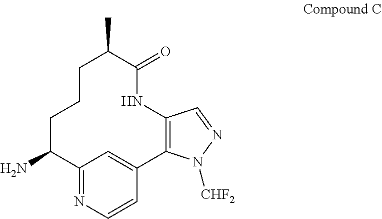 [SMILES: C[C@@H]1CCC[C@H](N)c2cc(ccn2)-c2c(cnn2C(F)F)NC1=O]

comprises the steps of:
(a) reacting Compound 17 having the structure:

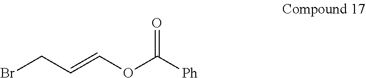 [SMILES: O=C(O/C=C/CBr)c1ccccc1]

with Compound 18 having the structure:

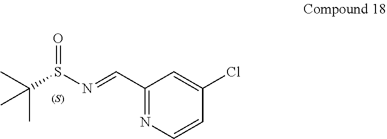

(b) subsequently adding a non-nucleophilic base followed by trimethylsilane chloride to afford the bis-hydrochloride salt of Compound 20 having the structure:

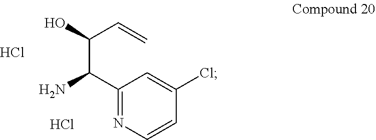

(c) reacting Compound 20 with a carbamate protecting group (PG) agent followed by propionic anhydride then a non-nucleophilic base to afford Compound 21 having the structure:

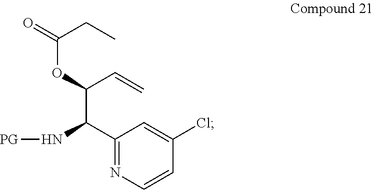

(d) reacting Compound 21 with a non-nucleophilic base to afford Compound 22 have the structure:

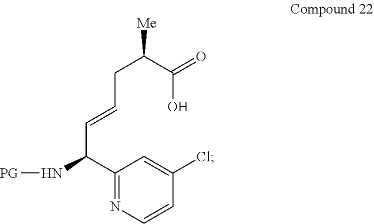

(e) reacting Compound 22 with a metal hydrogenation catalyst to afford Compound 23 having the structure:

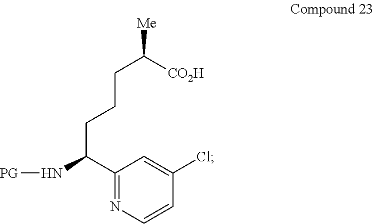

(f) reacting Compound 23 with 1-(difluoromethyl)-4-nitro-1H-pyrazole (Compound 12) and a metal catalyst to afford Compound 24 having the structure:

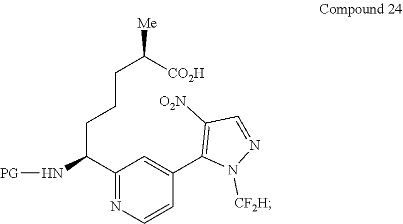

(g) reacting Compound 24 with a metal hydrogenation catalyst to afford Compound 25 having the structure:

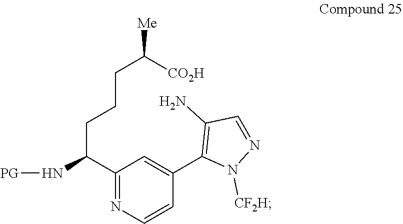

(h) reacting Compound 25 with a coupling agent to afford Compound 26 having the structure:

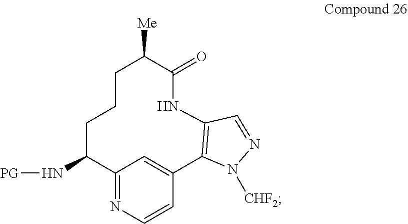

(i) reacting Compound 26 with an acid to afford Compound C:

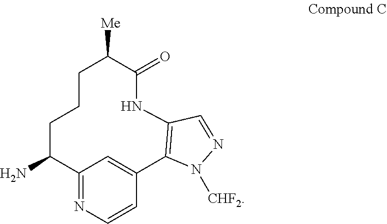 [SMILES: C[C@@H]1CCC[C@H](N)c2cc(ccn2)-c2c(cnn2C(F)F)NC1=O]

In an embodiment of the process for preparing Compound C, the non-nucleophilic base is selected from the group consisting of 1,8-diazabicyclo[5.4.0]undec-7-ene (DBU), lithium bis(trimethylsilyl)amide (LiHMDS), potassium bis(trimethylsilyl)amide (KHMDS), 1,5-diazabicyclo[4.3.0]non-5-ene (DBN), and N,N-Diisopropylethylamine (Hünig's base)

In an embodiment of the process for preparing Compound C, the carbamate protecting group (PG) agent of step (c) is selected from the group consisting of di-tert-butyl dicarbonate (Boc₂), carboxybenzyl chloride (Cbz-Cl), and methyl carbamate chloride (CH₃CO₂Cl).

In an embodiment of the process for preparing Compound C, the metal hydrogenation catalyst is selected from carbon-supported ruthenium, Crabtree's catalyst ([C₈H₁₂IrP(C₆H₁₁)₃C₅H₅N]PF₆), and carbon-supported palladium.

In an embodiment of the process for preparing Compound C, the metal catalyst for the reaction of Compound 12 in step (f) is selected from the group consisting of Pd(allyl)Cl]₂/X-Phos, XPHos Pd G3, and SPhos Pd G3.

In an embodiment of the process for preparing Compound C, the coupling reagent for the reaction of Compound 25 in step (h) is selected from the group consisting of 1,1′-carbonyldiimidazole (CDI), dicyclohexyl carbodiimide (DCC), 1-ethyl-3-(3-dimethylaminopropyl)carbodiimide (EDCI) and 1,1′-thiocarbonyldiimidazole (TCDI), and Chloro-N,N,N′,N′-tetramethylformamidinium hexafluorophosphate (TCFH).

In an embodiment of the invention, the present application provides a process for the preparation of Compound 11 via a lactic acid derivative as a starting material.

In an embodiment, the process for preparing Compound 11 having the structure:

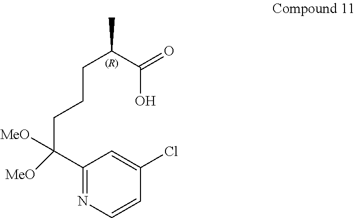

comprises the steps of:
(a) reacting Compound 27 having the structure:

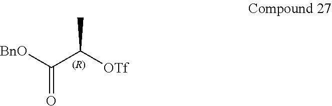

with (3-(1,3-dioxolan-2-yl)propyl)magnesium chloride to afford Compound 28 having the structure:

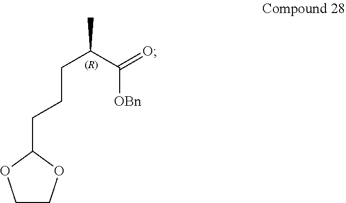

(b) reacting Compound 28 with an oxidizing agent to afford Compound 29 having the structure:

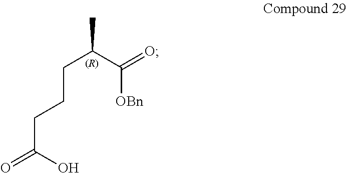

(c) reacting Compound 29 with N,O-dimethylhydroxylamine and a coupling agent to afford Compound 30 having the structure:

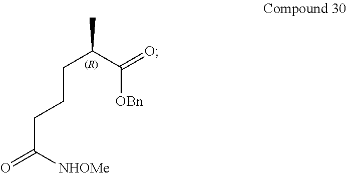

(d) reacting Compound 30 with 4-chloropyridin-2-yl magnesium bromide to afford Compound 31 having the structure:

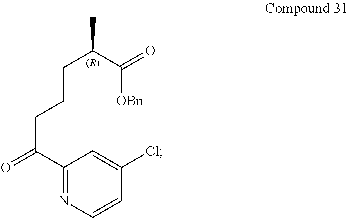

(e) subsequently adding a strong acid/alcoholic solution to afford Compound 32:

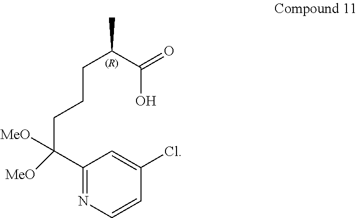

In an embodiment of the process for preparing Compound 11, the oxidizing agent in step (b) is selected from the group consisting of KMnO₄, Oxone, and NaClO₂.

In an embodiment of the process for preparing Compound 11, the strong acid in the strong acid/alcoholic solution in step (e) is selected from the group consisting of TMSCl, MeOH, MeOH/H₂SO₄, AcCl/MeOH.

In an embodiment of the process for preparing Compound 11, the alcohol solvent in the strong acid/alcoholic solution in step (e) is selected from the group consisting of methanol, ethanol, 2-isopropanol, n-propanol, n-butanol, and combinations thereof.

In an embodiment of the invention, the present application provides a process for the preparation of Compound 40 via a pseudoephedrine derivative as a starting material.

In an embodiment, the process for preparing Compound 40 having the structure:

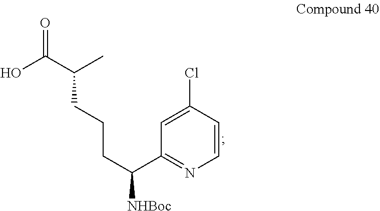 [SMILES: C[C@H](CCC[C@H](NC(=O)OC(C)(C)C)c1cc(Cl)ccn1)C(=O)O]

comprises the steps of
(a) reacting Compound 34 having the structure:

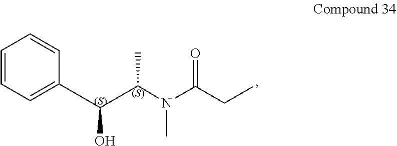

with 2-(3-bromopropyl)-1,3-dioxolane to afford Compound 35 having the structure:

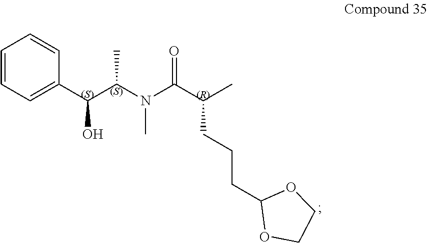

(b) reacting Compound 35 with strong base in a suitable solvent to afford Compound 36 having the structure:

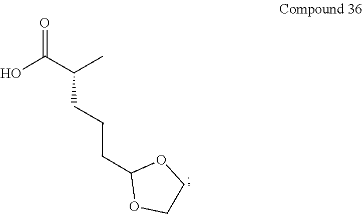

(c) reacting Compound 36 with benzyl alcohol and a coupling agent to afford Compound 37 having the structure:

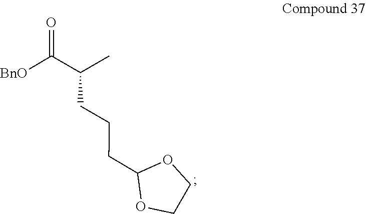 [SMILES: C[C@H](CCCC1OCCO1)C(=O)OCc1ccccc1]

(d) reacting a strong acid with Compound 37 followed by the addition of a chiral auxiliary with a metal catalyst to afford Compound 38 having the structure:

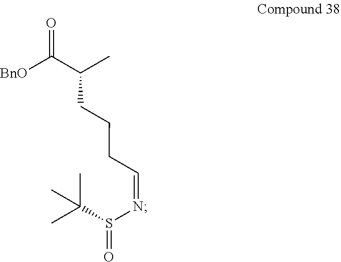

(e) reacting Compound 38 with (4-chloropyridin-2-yl)magnesium bromide to afford Compound 39 having the structure:

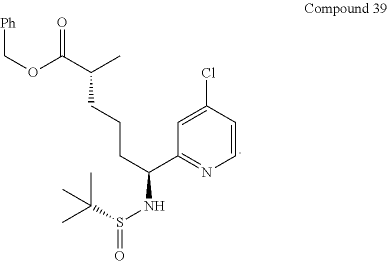

(f) hydrolyzing the ester followed by the addition of a protecting group to afford Compound 40:

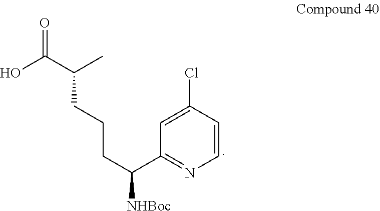 [SMILES: C[C@H](CCC[C@H](NC(=O)OC(C)(C)C)c1cc(Cl)ccn1)C(=O)O]

In an embodiment of the process for preparing Compound 40, the coupling agent of step (c) is selected from the group consisting of 1,1′-carbonyldiimidazole (CDI), dicyclohexyl carbodiimide (DCC), 1-ethyl-3-(3-dimethylaminopropyl)carbodiimide (EDCI) and 1,1′-thiocarbonyldiimidazole (TCDI).

In an embodiment of the process for preparing Compound 40, the suitable solvent in step (b) is selected from the group consisting of ethanol, methanol, tert-butanol, and combinations thereof.

In an embodiment of the process for preparing Compound 40, the metal catalyst in step (d) is selected from the group consisting of Ti(OEt)₄, CuSO₄, and Ti(OiPr)₄.

In an embodiment of the process for preparing Compound 40, the process further comprises a Lewis Acid in step (e) selected from ZrCl₄ and BF₃·OEt₂.

In an embodiment of the invention, the present application provides a process for the preparation of Compound 1 via a batch synthetic method.

In an embodiment, the process for preparing Compound 1 having the structure:

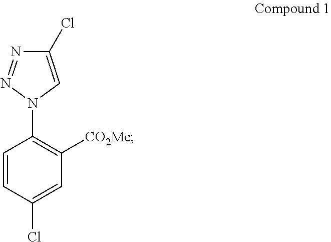 [SMILES: COC(=O)c1cc(Cl)ccc1-n1cc(Cl)nn1]

comprises the steps of:
(a) reacting Compound 4 having the structure:

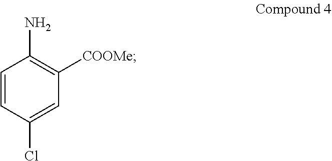 [SMILES: COC(=O)c1cc(Cl)ccc1N]

(i) with sodium nitrite and an acid a suitable solvent to form a first mixture; (ii) then adding the first mixture to solution comprising sodium azide in and a weak base to form a second mixture;
(iii) reacting the second mixture with trimethylsilylacetylene and copper (I) iodide (CuI) and a ligand to afford Compound 7 having the structure:

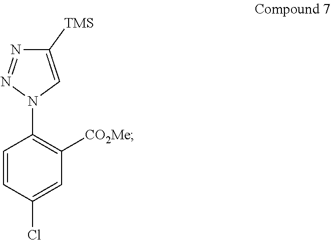 [SMILES: COC(=O)c1cc(Cl)ccc1-n1cc([Si](C)(C)C)nn1]

(b) chlorinating Compound 7 with a chlorinating agent to afford Compound 1:

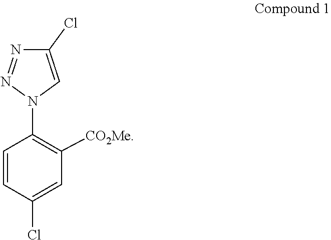 [SMILES: COC(=O)c1cc(Cl)ccc1-n1cc(Cl)nn1]

In an embodiment of the process for preparing Compound 1, the acid of step (a)(i) is selected from the group consisting of methane sulfonic acid, HBF₄, TsOH, H₂SO₄ and HCl.

In an embodiment of the process for preparing Compound 1, the weak base of step (a)(ii) is selected from the group consisting of sodium bicarbonate (NaHCO₃), potassium carbonate, pyridine, 2,6-lutidine, methylamine, triethylamine, and DMF.

In an embodiment of the process for preparing Compound 1, the ligand of step (a) (iii) is selected from the group consisting of tetramethylethlenediamine (TMEDA), NEt₂, DIPEA, TMEDTA, triethylamine, N,N-diisopropylethylamine, and N,N,N′,N″,N″-pentamethyldiethylenetriamine.

In an embodiment of the process for preparing Compound 1, the suitable solvent of step (a)(i) is selected from the group consisting of water, ionized water, DMF, ACN, and combinations thereof.

In an embodiment of the process for preparing Compound 1, the solvent in the aqueous solution of step (a)(ii) is selected from the group consisting of water, ionized water, DMF, ACN, and combinations thereof.

In an embodiment of the process for preparing Compound 1, the chlorinating agent of step (b) is selected from the group consisting of 1,3-dichloro-5,5-dimethylhydantoin, NCS, NaClO, and trichloroisocyanuric acid.

In an embodiment of the invention, the present application provides a process for the preparation of Compound 1 via a continuous flow synthetic method.

In an embodiment, the process for preparing Compound 1 having the structure:

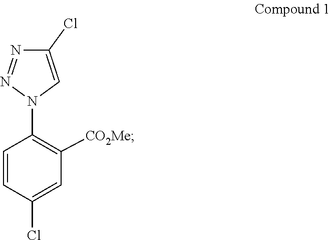 [SMILES: COC(=O)c1cc(Cl)ccc1-n1cc(Cl)nn1]

comprises the steps of:
(a) reacting Compound 4 having the structure:

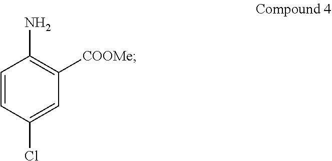 [SMILES: COC(=O)c1cc(Cl)ccc1N]

(i) with an acid and sodium nitrite in a suitable solvent;
(ii) reacting with a mixture of sodium azide and an aqueous weak base to form Compound 6 having the structure:

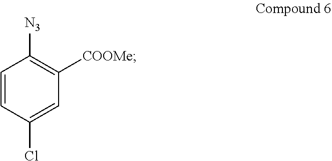 [SMILES: COC(=O)c1cc(Cl)ccc1N=[N+]=[N-]]

(b) subsequently coupling Compound 6 to chloroacetylene and a metal catalyst to afford Compound 1:

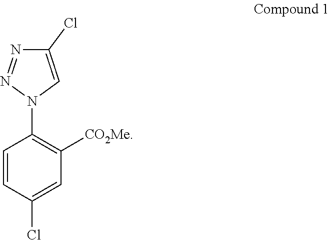 [SMILES: COC(=O)c1cc(Cl)ccc1-n1cc(Cl)nn1]

In an embodiment of the process for preparing Compound 1, the weak base of step (a)(ii) is selected from the group consisting of sodium bicarbonate (NaHCO₃), potassium carbonate, pyridine, 2,6-lutidine, methylamine, triethylamine, and DMF.

In an embodiment of the process for preparing Compound 1, the metal catalyst is selected from the group consisting of copper (I) iodide, CuBr, CuCl, Cu₂O, CuSO₄, CuSO₄ (5H₂O), Cu(OAc)₂, Cu(acac)₂, and CuCl₂.

In an embodiment of the process for preparing Compound 1, the ligan is selected from the group consisting of tetramethylethlenediamine (TMEDA), NEt₂, DIPEA, TMEDTA, triethylamine, N,N-diisopropylethylamine, and N,N,N′,N″,N″-pentamethyldiethylenetriamine.

In an embodiment of the process for preparing Compound 1, the acid of step (a)(i) is selected from the group consisting of methane sulfonic acid, HBF₄, TsOH, H₂SO₄ and HCl.

In an embodiment of the invention, the present application provides a process for the preparation of Compound 33.

In an embodiment, the process for preparing a compound having the structure:

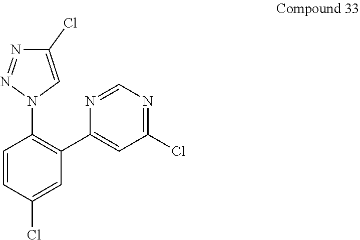 [SMILES: Clc1ccc(-n2cc(Cl)nn2)c(-c2cc(Cl)ncn2)c1]

comprises the steps of:
reacting Compound 32 having the structure:

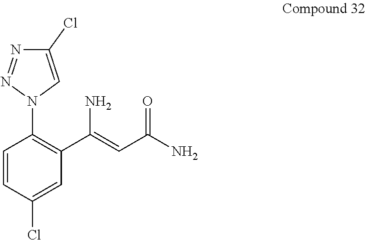 [SMILES: NC(=O)/C=C(\N)c1cc(Cl)ccc1-n1cc(Cl)nn1]

with ethylformate to afford Compound 33:

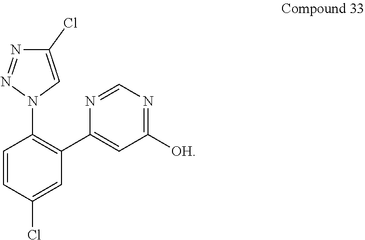 [SMILES: Oc1cc(-c2cc(Cl)ccc2-n2cc(Cl)nn2)ncn1]

In an aspect, the invention provides a compound selected from the group consisting of:

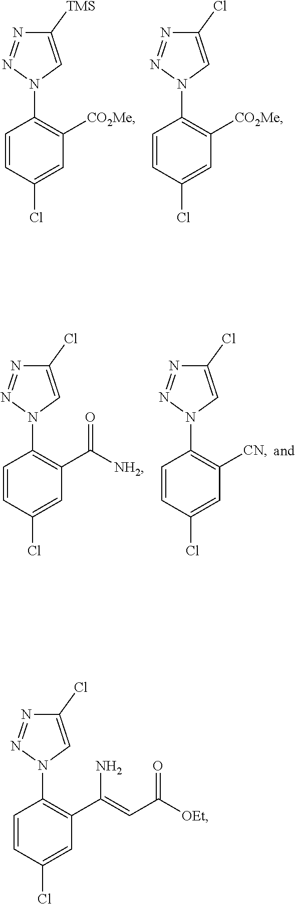

or a stereoisomer or a tautomer thereof.

### EXAMPLES

The following examples are offered for purposes of illustration, and are not intended to limit the scope of the claims provided herein. All literature citations in these examples and throughout this specification are incorporated herein by references for all legal purposes to be served thereby.

General aspects of these exemplary methods are described in the schemes and the Examples. Each of the products of the following processes is optionally separated, isolated, and/or purified prior to its use in subsequent processes.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. (Protective Groups in Organic Synthesis, 4^{th} Ed., Wiley-Interscience (2006)).

All reactions were performed under nitrogen unless specified. Solvents for extractions and reactions were ACS reagent grade without purification. ¹H NMR and ¹³C NMR spectra were recorded on a Bruker Advance DRX 400 spectrometer, a Bruker Advance III 400 spectrometer, a Bruker Advance 500 MHz spectrometer and a Bruker Advance III 400 spectrometer. Chemical shifts are given in ppm (δ); multiplicities are indicated by s (singlet), d (doublet), t (triplet), q (quartet), m (multiplet) or br (broadened). Coupling constants, J, are reported in Hertz. Solvent removal was affected by rotary evaporation under vacuum (˜25-40 mmHg).

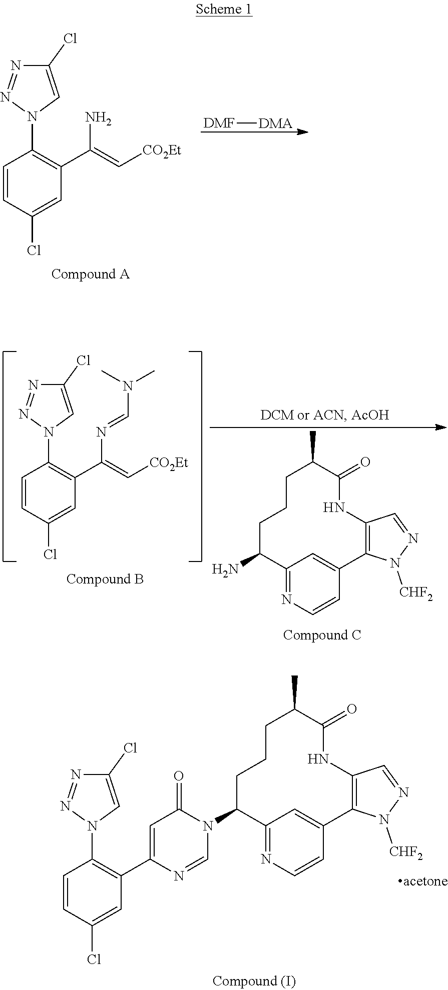

### Example 1: Synthesis of Compound (I)·acetone

Step (a): Coupling Reaction

A 1 L reactor was charged with ethyl (Z)-3-amino-3-(5-chloro-2-(4-chloro-1H-1,2,3-triazol-1-yl)phenyl)acrylate (Compound A) (1.07 eq., 104.4 g), acetonitrile (6.8 L/kg, 680 mL) and N,N-dimethylformamide dimethyl acetal (1.26 eq, 50.1 mL) and the resulting solution was heated to 75° C. for 30 minutes followed by atmospheric distillation of the methanol by-product. The reaction was cooled to 30° C. prior to the addition of acetic acid (10 eq. 170.9 mL), and stirred at 40° C. for 5 hours. At 30° C., (5R,9S)-9-amino-2¹-(difluoromethyl)-5-methyl-2¹H-3-aza-1(4,2)-pyridina-2(5,4)-pyrazolacyclononaphan-4-one (Compound C) (1.0 eq., 100 mL) and acetonitrile (2.5 L/kg, 250 mL) were charged and then heated to 65° C. for 2 hours. Reaction is cooled to 40° C., then 254 mL (6.1 eq.) triethylamine is added.

Step (b): Work-Up

Acetonitrile was replaced by trimethylamine by co-distillation under vacuum (200 mbar). Distilled 355 mL (3.55 L/kg), then charged 145 mL (3.5 eq.) triethylamine. Distilled 240 mL (2.4 L/kg) at 200 mbar, then charged 145 mL (3.5 eq.) triethylamine. Finally distilled 225 mL (2.25 L/kg) at 200 mbar, then charged 112 mL (2.7 eq.) triethylamine.

Step (c): Filtration

At 45° C., water (0.32 L/kg, 32 mL) and acetone (4.4 L/kg, 440 mL) were charged. The reaction was then cooled to 25° C. follow by filtration over charcoal and rinsed with 65 mL (0.65 L/kg) acetone.

Step (d): Crystallization

At 45° C., methanol (0.68 L/kg, 68 mL) and water (0.59 L/kg, 59 mL) were charged followed by 1.0 wt. % (1 g) seeding material. The mixture was stirred for 2 hours. Water (140 mL (1.24 L/kg)) was dosed over 4 hours. Another 275 mL (2.75 L/kg) of water was dosed over 3 hours. The reaction mixture was then cooled to 10° C. over 4 hours and kept overnight prior to filtration. The wet cake was rinse with a 570 mL mixture of acetone water (47/53) and dried under vacuum at 50° C. for 16 hours. Compound (I) was obtained in 89-95% yield (>99.5% purity) as an acetone solvate.

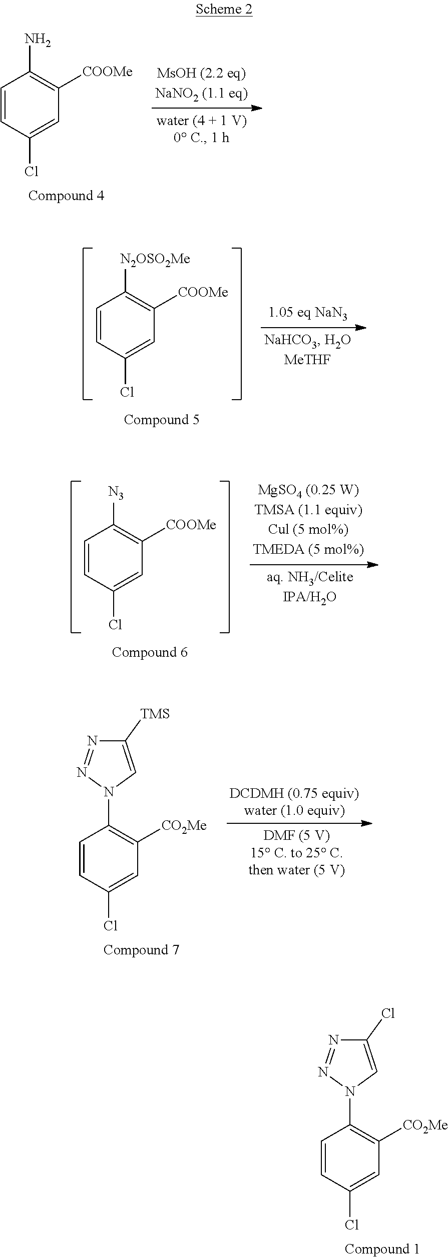

### Example 2: Synthesis of Compound 1—Batch Synthesis Method

Step (a): Preparation of the Diazo Intermediate (Compound 5)

A 50 L reactor was charged with water (18 L, 3.6 L/kg), methyl 2-amino-5-chlorobenzoate (Compound 4) (5.00 kg, 1 equiv.) and the resulting slurry was cooled down to 0°-5° C. 70% aqueous methane sulfonic acid (8.14 kg, 2.2 equiv.) was charged over 1 hour while keeping the temperature at 0°-5° C. A solution containing sodium nitrite (2.05 kg, 1.1 equiv.) and water (5.25 kg, 1.05 V) was dosed to the reaction mixture over 4 hours while keeping the temperature at 0°-5° C. The resulting slurry was stirred at 0°-5° C. for 8 hours.

### Step (b): Preparation of the Azide Intermediate (Compound 6)

A 100 L reactor was charged with water (20 L, 4 L/kg), sodium bicarbonate (0.91 kg, 0.4 equiv.), sodium azide (1.8 kg, 1.0 equiv.), 2-methyltetrahydrofuran (21.5 kg, 5.0 V) and the resulting mixture was stirred at 20° C. for 30 min. The diazo solution of step a) was added to this solution over 4 hours while keeping the temperature at 20°-25° C. and the mixture was aged 2 hours at that temperature. The biphasic mixture was settled and the two layers were separated. The organic layer was washed twice with 18.6 w % aqueous sodium chloride solution (12.3 kg, 2 kg/kg). Magnesium sulfate (1.43 kg, 0.285 kg/kg) was added to the azide solution in MeTHF and stirred for 1 hour.

Step (c): Preparation of Compound 7

N,N,N′,N′-tetramethylethylenediamine (0.16 kg, 0.05 equiv.), copper iodide (0.26 kg, 0.05 equiv.) and trimethylsilylacetylene (2.91 kg, 1.1 equiv.) were charged to the reactor at 20-25° C. and the reaction mixture was heated to 45° C. over 1 hour and stirred at that temperature for 10 hours. After reaction completion, the mixture was filtered and the filter cake was washed twice with 2-methyltetrahydrofuran (2×2.6 kg, 2×0.6 V).

In a separate reactor, a solution containing sodium chloride (2.9 kg), water (10.0 kg) and 20 wt. % aqueous ammonia (9.2 kg) was prepared.

11.0 kg of that solution was added to the filtrate (2.0 V) and the resulting mixture was stirred at 20-25° C. for 30 min, filtered over celite (1.25 kg, 0.25 kg/kg) and the filter cake was washed twice with 2-methyltetrahydrofuran (2×2.6 kg, 2×0.6 V). The biphasic mixture was then settled, and the two layers were separated. The organic layer was washed again with the aqueous sodium chloride/ammonia solution (11.0 kg, 2.0 V) and once more with saturated sodium chloride aqueous solution (8.5 kg, 1.5 V). The 2-methyltetrahydrofuran solution containing crude methyl 5-chloro-2-(4-(trimethylsilyl)-1H-1,2,3-triazol-1-yl)benzoate (Compound 7) was then concentrated to 2.5 V (12.5 L) under vacuum. 2-propanol (11.8 kg, 3.0 V) was charged to the mixture which was heated to 60°-65° C. over 30 min. Once all the compound dissolved, the solution was cooled down to 0°-5° C. over 4 hours and water (32.5 kg, 6.5 V) was charged over 3 hours at 0°-5° C. and aged at that temperature for 2 hours. After filtration, and the wet cake was washed with a 2-propanol/water mixture (14.75 kg, 50/50 v/v ratio) and dried under vacuum at 40° C. for 12 hours. Methyl 5-chloro-2-(4-(trimethylsilyl)-1H-1,2,3-triazol-1-yl)benzoate (Compound 7) was obtained in 85-88% yield, >99.5% purity and assay. ¹H NMR (CDCl₃, 300 MHz): δ=7.87 (d, J=2.4 Hz, 1H), 7.77 (s, 1H), 7.55 (dd, J=8.5, 2.4 Hz, 1H), 7.38 (d, J=8.4 Hz, 1H), 3.61 (s, 3H), 0.32 (s, 9H) ppm. ¹³C NMR (CDCl₃, 75 MHz): δ=164.4, 146.3, 135.3, 134.4, 132.3, 130.8, 130.5, 128.6, 127.7, 52.5, −1.3 ppm.

Step (d): Preparation of Compound 1

A 50 L reactor was charged with DMF (33.3 kg, 5 L/kg), Compound 7 (1 equiv., 7.1 kg), water (0.41 kg, 1.0 equiv.) and the resulting solution was cooled down to 0-5° C. 1,3-Dichloro-5,5-dimethylhydantoin (3.4 kg, 0.75 equiv.) was charged in portions over 6 hours while keeping the temperature below 10° C. and the reaction was stirred for 15 additional hours at 0-5° C. After reaching full conversion, the reaction mixture was heated to 30° C. within 2 hours and aged at that temperature for 2 hours.

A separate reactor was charged with water (35.3 kg, 5 L/kg) and heated to 30° C. The reaction mixture was then dosed to this second reactor over 5 hours while controlling the temperature between 30-35° C. After 20 min aging, the slurry was cooled to 15-20° C. over 2 hours and aged at that temperature for 2 hours. After filtration, the wet cake was washed with methanol/water (1 L/kg methanol, 1.2 L/kg water) and dried under vacuum at 40° C. for 16 hours. Methyl 5-chloro-2-(4-chloro-1H-1,2,3-triazol-1-yl)benzoate (Compound 1) was obtained in 89-93% yield, >99.5% purity and assay.

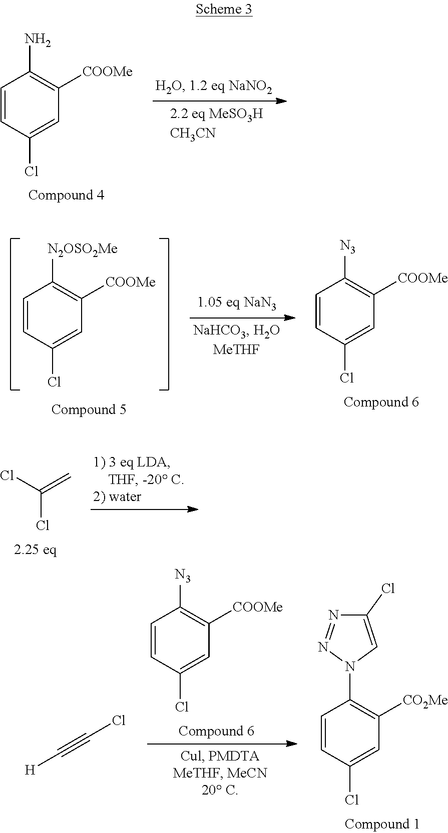

### Example 3: Synthesis of Compound 1—Continuous Flow Method

Step (a): Preparation of the Diazo Intermediate

A solution of methyl 2-amino-5-chlorobenzoate (Compound 4) (741.0 g, 1 equiv.), methane sulfonic acid (844 g, 2.2 equiv.), water (2223 g) and acetonitrile (1747 g) was mixed in a continuous flow reactor with a solution of sodium nitrite (331 g, 1.2 equiv.) and water (2964 g) at −0° C.

Step (b): Preparation of the Azide Intermediate

The diazonium solution of step a) was reacted with a mixture of water (2964 g), sodium bicarbonate (134 g, 0.4 equiv.), sodium azide (272 g, 1.05 equiv.), and 2-methyltetrahydrofuran (2549 g) at room temperature. The aqueous layer was discarded. The organic stream was used directly in the next step (in situ yield 99%).

Step (c): Preparation of the Chloroacetylene

A mixture of 1,1-dichloroethene (871 g, 2.25 eq) in THF (1635 g) was mixed (at −20° C.) with an LDA solution (2.0M, 2431 g, 3.0 eq) diluted with THF (3257 g). The reaction was quenched with water (5988 g) and the aqueous layer was discarded. The organic layer was used directly in the next step.

Step (d): Preparation of Compound 1

The aryl azide solution of step (b) was first mixed with a solution of N,N,N′,N′-tetramethylethylene-diamine (101 g, 0.22 equiv.), copper iodide (38 g, 0.05 equiv.), and THF (1159 g). The azide-copper solution was then mixed with the chloroacetylene solution of step c) at 60° C. The reaction mixture was quenched with a solution of ammonium chloride in ammonium hydroxide. The layer was separated and the aqueous layer was back extracted with THF. The combined organic layers were washed with a brine solution. The aqueous layer was back extracted and the organic layers were combined. THF was removed by distillation and replaced by MeOH. The product was crystallized by heating to 60° C., cooling to 15° C., and by adding water, yielding Compound 1. (92.6% overall yield, purity >99%).

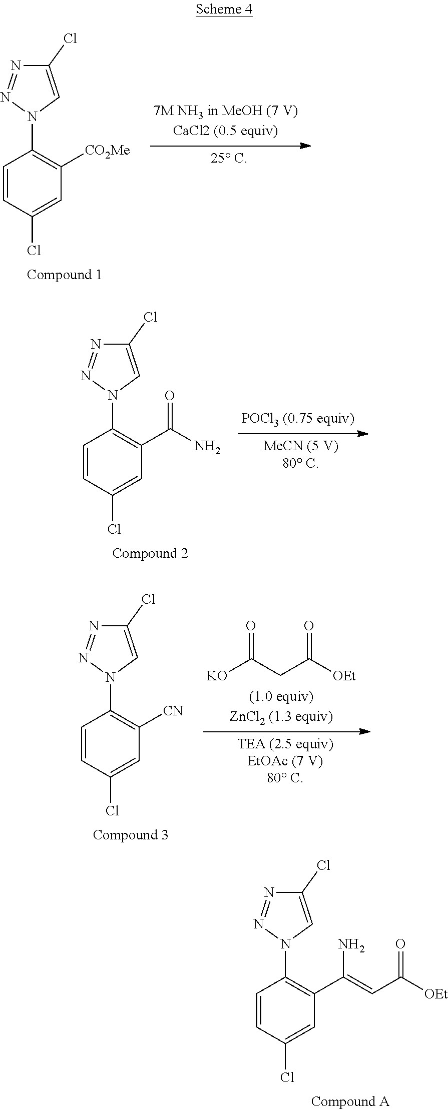

### Example 3a: Synthesis of Compound 1—Continuous Flow Method

Step 1: Procedure for Continuous Flow Synthesis of Compound 6

Flow Parameter Settings:

| | | | | Flow rate |
|---|---|---|---|---|
| | d | Amount | Equiv./V | (g/min) |
| Compound 4 solution: | | 562.3 | | 6 |
| Compound 4 | | 75.0 | 1 | |
| MSA | 1.480 | 85.4 | 2.2 | |
| Water | 1.000 | 225 | 3 V | |
| ACN | 0.786 | 177 | 3 V | |
| Sodium nitrite solution: | | | | 1.78 |
| NaNO2 | | 40.0 | 1.2 | |
| Water | 1.000 | 160 | 4 V | |
| Sodium azide solution: | | | | 2.84 |
| Water | 1.000 | 285 | 3 V | |
| NaHCO3 | | 17.2 | 0.4 | |
| NaN3 | | 35.0 | 1.05 | |
| MeTHF | 0.854 | 256 | 4 V | 3.2 |

A continuous flow reactor was precooled to 0° C. using a recirculating chiller. Equilibration of continuous flow reactor was started by setting aniline (Compound 4) and NaNO₂ flowrates to 6 g/min and 1.78 g/min respectively while diverting the effluent to waste. After about 1 minute the effluent from the reactor turned from a deep red color to a pale-yellow color indicating full equilibration of the reactor. The 2-Me-THF and NaN₃ streams were started with flowrates of 3.2 g/min and 2.84 g/min respectively while diverting the streams to waste. Once each pump had reached the setpoints, all valves were switched to divert flows to the quench reactor loop. Rapid evolution of N₂ (gas) was immediately observed. Effluent from the quench reactor was diverted to waste until about 1 minute had passed. The effluent from the quench reactor was then diverted to the settler unit. Once the organic phase in the settler had reached the dip tube for the organic collection pump, the collection pump was started and set to a rate to maintain a constant volume within the settler. Organic effluent was collected for a total of 50 minutes. A total of 266 g of organics were collected. ¹H-NMR analysis of the organics showed the solution to be 17.02 wt. % of Compound 6. Assay yield: 45.3 g, 99% yield. HPLC analysis showed the solution to be >99A % of Compound 6.

Step 2: Procedure for the Continuous Flow Synthesis of Compound 1

Under nitrogen, THF (300 mL) was added to CuI (5.71 g, 30 mmol). Then PMDTA (5.20 g, 30 mmol) was added and the mixture was sonicated to promote full dissolution. (CuI solution)

Flow Parameters Settings

| | | density | | | | |
|---|---|---|---|---|---|---|
| Reagent | equiv | (g/mL) | mmol/mL | conc (wt. %) | mL/min | g/min |
| Compound 6 solution | 1 | 0.92 | 0.713 | 0.164 | 2.071 | 1.91 |
| Chloroacetylene solution | 1.05 | 0.83 | 0.332 | 0.0242 | 4.666 | 3.87 |
| CuI solution | 0.05 | 0.897 | 0.1 | | 0.763 | 0.684 |
| Total | | | | | 7.5 | 6.46 |

In a continuous flow reactor, delivery of chloroacetylene solution (3.87 g/min), Compound 6 solution (1.91 g/min), and CuI solution (0.763 mL/min) were initiated sequentially. Material was flowed through the system for two residence volumes (10 minutes) before collection was started. One stoppage was performed during the run, prior to which effluent from the plug flow reactor (PFR) was diverted to waste. Restarting the system was performed similar to that described above, and the system was allowed to re-equilibrate for 2 residence volumes (10 minutes) prior to collection of material in the quench vessel. In total, material was collected for 131 minutes, corresponding to 193.4 mmol of Compound 6 consumed. The material from the quench vessel was transferred to a separatory funnel and the organic phase was separated. The aqueous phase was extracted with additional THF (155 mL, 3.8 volumes). The combined organic phases were then washed with 50% brine solution (155 mL, 3.8 volumes), and the resulting aqueous phase was extracted with additional THF (78 mL, 1.9 volumes). The combined organic phases were then analyzed by ¹H NMR spectrometry using 1,3,5-trimethoxybenzene as an internal standard to establish the potency of Compound 1. In total, 871.6 g of organics were collected. A small sample (ca. 20 mL) was removed for additional analysis and remaining material was concentrated under reduced pressure to provide 54.7 grams of a tan solid, which was found to be 95% Compound 1 by quantitative NMR analysis, with 5% residual ethylbenzene remaining.

Isolation: Solvent was removed by distillation to 5 L/kg and cooled to 35° C. After seeding, heptane (11 L/kg) was added and the reaction mixture was cooled to 10° C. After filtration Compound 1 was obtained as a white solid (90% yield).

### Example 4: Synthesis of Ethyl (Z)-3-amino-3-(5-chloro-2-(4-chloro-1H-1,2,3-triazol-1-yl)phenyl)acrylate (Compound A)

A 50 L reactor was charged with 7 mol/L ammonia in methanol (30.2 kg, 7 L/kg), anhydrous calcium chloride (1.13 kg, 0.5 equiv.), methyl 5-chloro-2-(4-chloro-1H-1,2,3-triazol-1-yl)benzoate (Compound 1) (5.7 kg, 1.0 equiv.) and the resulting slurry was stirred at 25° C. for 20 hours. After reaction completion, the mixture was concentrated to 3 L/kg total volume and the resulting slurry was then heated to reflux until full dissolution. Water (27.7 kg, 5 L/kg) was dosed at 60° C. over 3 hours and the mixture was cooled to 0°-5° C. over 4 hours. After filtration, the wet cake was washed with methanol/water (0.75 L/kg methanol, 1.25 L/kg water) and dried under vacuum at 40° C. for 20 hours. 5-Chloro-2-(4-chloro-1H-1,2,3-triazol-1-yl)benzamide (Compound 2) was obtained in 92-95% yield, >99.5% purity and assay. ¹H NMR (DMSO-d₆, 300 MHz): δ=8.71 (s, 1H), 8.12-8.05 (br s, 1H), 7.80-7.73 (m, 2H), 7.72-7.66 (m, 1H), 7.66-7.59 (br s, 1H) ppm.

A 50 L reactor was charged with acetonitrile (8.5 kg, 3 L/kg) and Compound 2 (3.7 kg, 1.0 equiv.). Phosphoroxy chloride (1.6 kg, 0.75 equiv.) was then dosed over 20 min and the resulting slurry was heated to 75°-80° C. over 90 min and aged at that temperature for 5 hours. After reaction completion, the mixture was cooled down to 60°-65° C. and water (17.0 kg, 4.7 L/kg) was added over 1 hour. After further cooling to 50°-54° C., 15% aqueous sodium hydroxide (6.4 kg, 1.8 kg/kg) was dosed over 1 hour. The resulting slurry was cooled to 20°-25° C. over 2 hours. After filtration, the wet cake was washed twice with water (2×1 L/kg) and dried under vacuum at 50° C. for 19 hours. 5-Chloro-2-(4-chloro-1H-1,2,3-triazol-1-yl)benzonitrile (Compound 3) was obtained in 95-98% yield, >99.5% purity and assay. ¹H NMR (DMSO-d₆, 300 MHz): δ=9.06 (s, 1H), 8.38 (d, J=2.4 Hz, 1H), 8.08 (dd, J=8.5, 2.4 Hz, 1H), 7.94 (d, J=8.4 Hz, 1H) ppm. ¹³C NMR (DMSO-d₆, 75 MHz): δ=136.2, 135.2, 134.8, 134.5, 134.2, 127.5, 123.8, 114.4, 108.9 ppm.

A 30 L reactor was charged with ethyl acetate (6.1 kg, 5 L/kg), Compound 3 (1.6 kg, 1.0 equiv.), potassium ethyl malonate (2.25 kg, 2.0 equiv.), triethylamine (1.67 kg, 2.5 equiv.) and zinc chloride (1.13 kg, 1.25 equiv.). The resulting mixture was heated to 75-78° C. over 2 hours and aged at that temperature for 20 hours. After reaction completion, the mixture was cooled down to 20°-25° C. and 15% ammonia in water (7.5 kg, 5.2 L/kg) was charged over 1 hour. After addition of celite (0.32 kg, 0.2 kg/kg), the mixture was filtered and the wet cake was washed twice with ethyl acetate (2×2.85 kg, 2 L/kg). After separation of the two layers, the organic layer was concentrated to 3.1 L/kg total volume, ethanol (7.5 kg, 6 L/kg) was charged and the mixture was concentrated to 4.1 L/kg total volume. The resulting slurry was then heated to 60° C. until full dissolution, water (4.75 kg, 3 L/kg) was dosed at 60° C. over 4 hours and the mixture was cooled to 0-5° C. over 4 hours and aged at that temperature for 4 hours. After filtration, the wet cake was washed with ethanol/water (0.8 L/kg MeOH, 1.2 L/kg water) and dried under vacuum at 40° C. for 20 hours. Ethyl (Z)-3-amino-3-(5-chloro-2-(4-chloro-1H-1,2,3-triazol-1-yl)phenyl)acrylate (Compound A) was obtained in 92-95% yield, >99.5% purity and assay. ¹H NMR (DMSO-d₆, 300 MHz): δ=8.63 (s, 1H), 7.86-7.50 (m, 3H), 7.50-7.02 (br s, 1H), 4.24 (s, 1H), 3.99 (q, J=7.1 Hz, 2H), 1.14 (t, J=7.1 Hz, 3H) ppm. ¹³C NMR (DMSO-d₆, 75 MHz): δ=168.5, 156.4, 135.5, 134.9, 133.6, 132.8, 130.3, 129.7, 128.3, 124.3, 84.4, 58.2, 14.4 ppm.

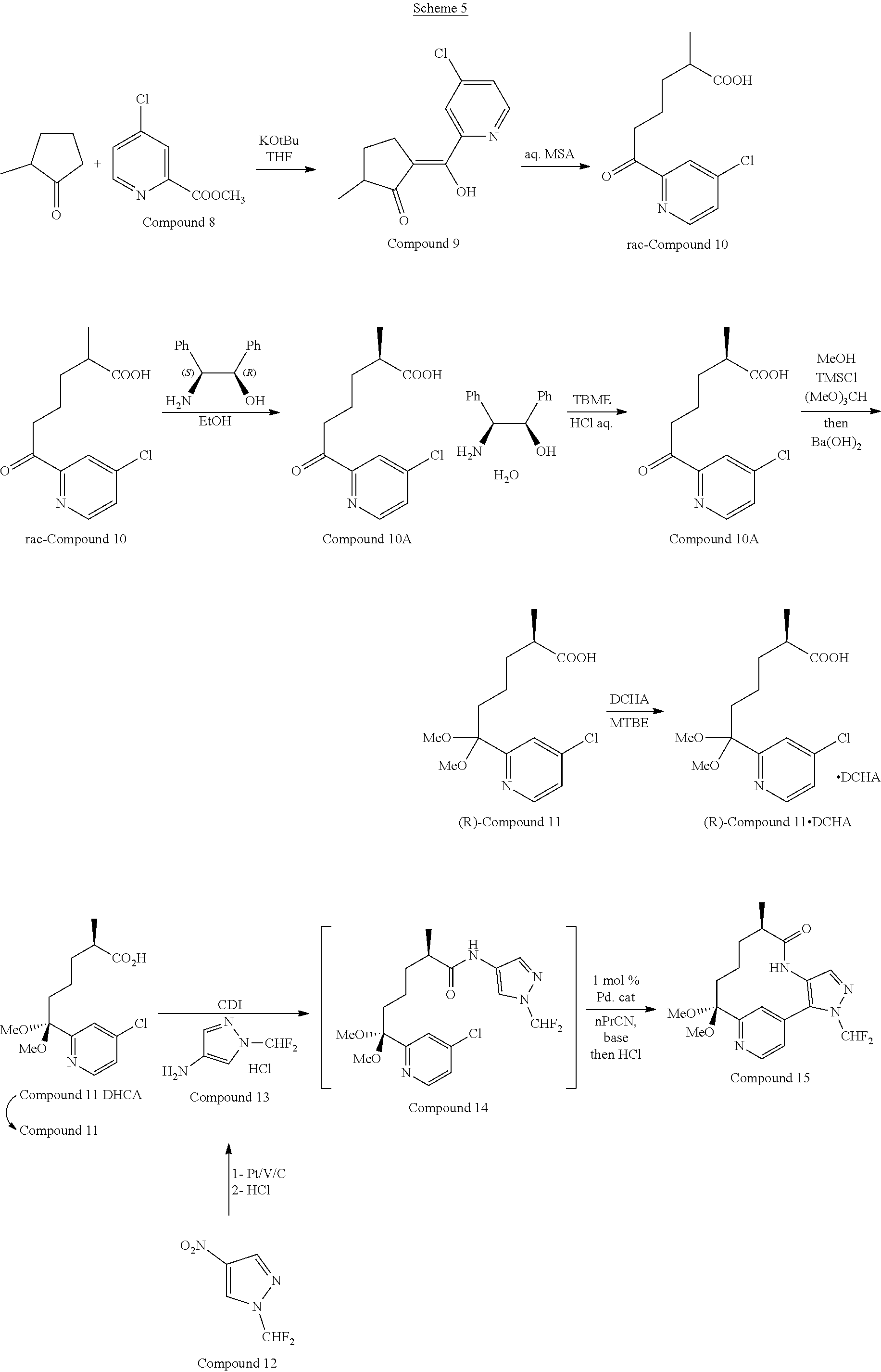

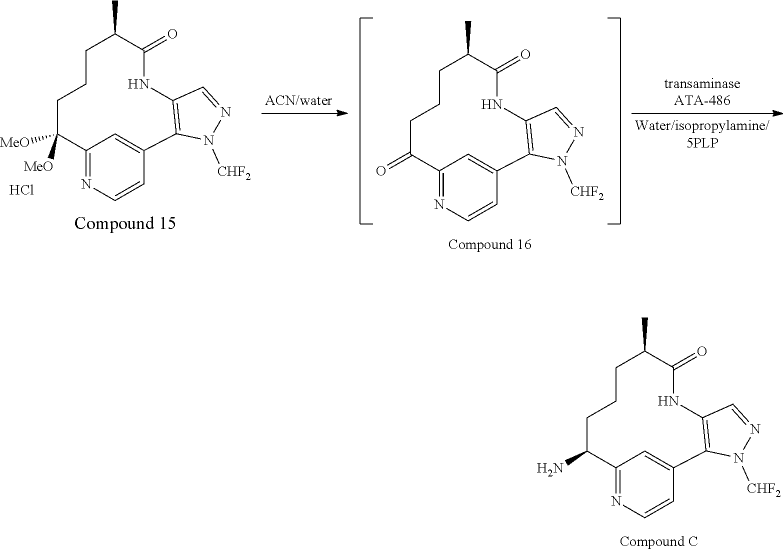

### Example 5: Synthesis of Macrocycle “C”

To a slurry of a mixture of 2-methylcyclopentanone (93.30 g, 931.7 mmol, 98 mass %), methyl 4-chloropicolinate (Compound 8) (158.02 g, 902.55 mmol) in THF (1500 mL, 18400 mmol) was added potassium tert-butoxide (1 mol/L) in THF (1200 g, 1330 mmol, 1 mol/L) at −30° C. The resulting yellow slurry was stirred at between −24° C. to 30° C. for 1 h. In a separate 4 L rector was charged with sulfuric acid (13.14 mol/L) in water (92 g, 660.2 mmol, 13.14 mol/L) and water (800 g, 44407.9 mmol) and was precooled to 0° C. The yellow slurry containing potassium (Z)-(4-chloropyridin-2-yl)(3-methyl-2-oxocyclopentylidene)methanolate (anion of Compound 9) was poured into the cold acid solution and resulted in a slurry. THF was distilled off at 15° C. with jacket set at 45° C. under vacuum, 115 mbar. To the slurry (˜1 Liter) was added 500 mL of water. The precipitated solids were collected and the aqueous was discarded. The collected solids were charged back to the reactor along with 320 mL of MSA and 1 Liter of water. The slurry was heated to 65° C. and all the solids were dissolved after 60 minutes. The dark solution was held for 3 h at 65° C. before it was cooled to rt then 0° C. A slurry was formed and then was filtered. The residue was collected and dried at room temperature, a total amount of 139.8 g beige colored 6-(4-chloropyridin-2-yl)-2-methyl-6-oxohexanoic acid (rac-Compound 10) obtained. The filtrate was charged back to the reactor and the pH was adjusted to 5.1 with 28 wt. % NH₄OH. Solids were formed during the pH adjustment and was filtered at room temperature. Additional 41 g of off-white 6-(4-chloropyridin-2-yl)-2-methyl-6-oxohexanoic acid (rac-Compound 10) was obtained. ¹H NMR (400 MHz, DMSO-d₆): δ 12.07 (1H, s), 8.70 (d, J=5.31 Hz, 1H), 7.94 (dd, J=7.94, 1.77 Hz, 1H), 7.82 (dd, J=5.18, 2.15 Hz, 1H), 3.15 (m, 2H), 2.35 (m, 1H), 1.60 (m, 3H), 1.42 (m, 1H), 1.05 (d, J=6.82 Hz, 3H). LRMS: [C₁₂H₁₄ClNO₃₊ H]⁺, 258.24, 256.25.

### Example 6: Chiral resolution of rac-6-(4-chloropyridin-2-yl)-2-methyl-6-oxohexanoic acid (rac-Compound 10) by fractional crystallization of its (R)-enantiomer with (1R,2S)-erythro-2-amino-1,2-diphenylethanol

487 g (1.905 mol) of rac-6-(4-chloropyridin-2-yl)-2-methyl-6-oxohexanoic acid (rac-Compound 10) and 223 g (1.048 mole) of (1R,2S)-erythro-2-amino-1,2-diphenylethanol were dissolved in 4.8 kg of ethanol (96% by volume) at a temperature of 70° C. The solution was allowed to slowly cool to r.t. and afterwards cooled to 0° C. The formed precipitate was filtered off, washed with cold ethanol (96% by volume) and dried to afford (R)-6-(4-chloropyridin-2-yl)-2-methyl-6-oxohexanoic acid·(1R,2S)-erythro-2-amino-1,2-diphenylethanol·H₂O, i.e. the diastereomeric salt of (R)-6-(4-chloropyridin-2-yl)-2-methyl-6-oxohexanoic acid (Compound 10A) and (1R,2S)-erythro-2-amino-1,2-diphenylethanol in the form of its monohydrate, as white needles. Yield: 407 g, 91% (based on the original amount of (R)-6-(4-chloropyridin-2-yl)-2-methyl-6-oxohexanoic acid). S/R ratio: 19.1:80.9.

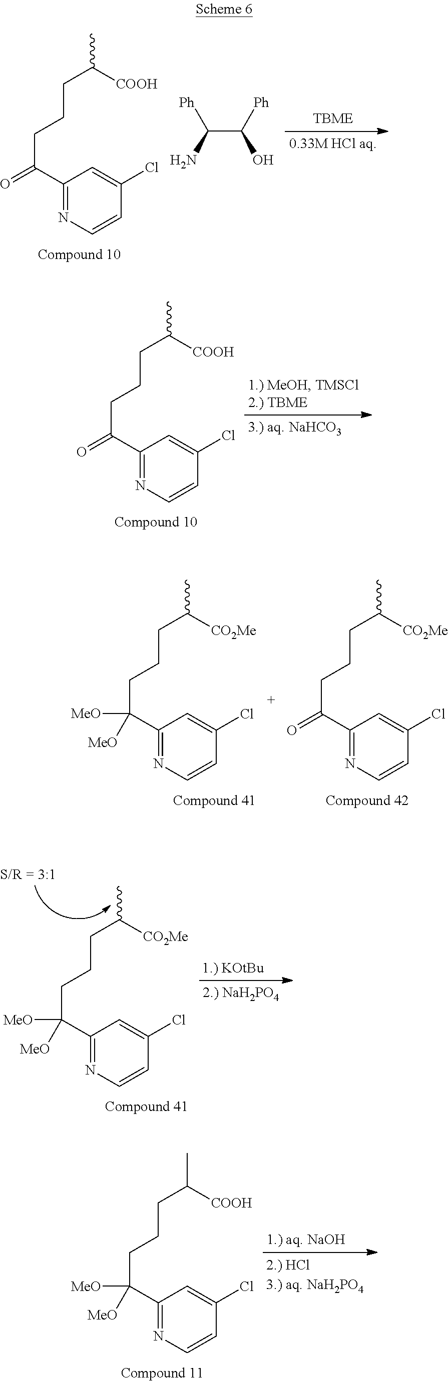

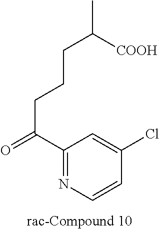 [SMILES: CC(CCCC(=O)c1cc(Cl)ccn1)C(=O)O]

### Example 7: Racemization of the 6-(4-chloropyridin-2-yl)-2-methyl-6-oxohexanoic acid contained in the mother liquor resulting from the fractional crystallization of Example 6

Step 1: Protection of 6-(4-chloropyridin-2-yl)-2-methyl-6-oxohexanoic acid

The mother liquor from the fractional crystallizations of Example 6 was concentrated and dried. The obtained residue was treated with a mixture of TBME (tert-butylmethyl ether) and a 0.33M aqueous solution of hydrochloric acid and then the organic phase was separated, dried and concentrated in analogy to the procedure of Example 9, in order to remove remaining 2-amino-1,2-diphenyl ethanol. 639 g (2.50 mol) of the 6-(4-chloropyridin-2-yl)-2-methyl-6-oxohexanoic acid (Compound 10) obtained this way were dissolved in 1.95 kg of dry methanol. To this solution was added 271 g (2.50 mol) of trimethylsilyl chloride. The reaction mixture was stirred at room temperature for 12 hours. Afterwards 1.95 kg of TBME was added and the pH of the mixture was neutralized with saturated aqueous sodium hydrogen carbonate (NaHCO₃). The organic phase was separated, washed with brine, dried over sodium sulfate and concentrated in vacuo to yield the dried product which contained 6-(4-chloropyridin-2-yl)-2-methyl-6,6-dimethoxyhexanoic acid methyl ester (Compound 41) as the main product and 6-(4-chloropyridin-2-yl)-2-methyl-6-oxohexanoic acid methyl ester (Compound 42) as a side product. Yield: 587 g (about 87%).

Step 2: Racemization

587 g (about 1.86 mol) of the product obtained in step 1, which had an S/R ratio of approximately 75:25, were dissolved in 3.34 kg of dry toluene and the solution was cooled to 0° C. 104 g (0.93 mol) of potassium tert-butoxide were added and the reaction mixture was stirred for 12 hours at r.t. Afterwards the mixture was cooled to 0° C. and 1 kg of saturated aqueous NaH₂PO₄ were added. The organic phase was separated, washed with 1 kg brine and concentrated in vacuo to afford the racemized product (Compound 11), which may contain up to 10% by weight of the tert-butyl ester of 6-(4-chloropyridin-2-yl)-2-methyl-6,6-dimethoxyhexanoic acid, as determined by ¹H-NMR. Yield: 500 g (about 85%).

Step 3: Preparation of racemic 6-(4-chloropyridin-2-yl)-2-methyl-6-oxohexanoic acid by deprotecting the racemate obtained in step 2

500 g (about 1.58 mol) of the racemic product of step 2 were dissolved in 2.5 kg THF and 158 g sodium hydroxide (3.96 mol) dissolved in 1.58 kg water were added. The solution is stirred at room temperature for 12 hours. Then, 524 ml of concentrated hydrochloric acid (6.33 mol) were slowly added. After complete conversion (about 12 hours), 300 g NaH₂PO₄ dissolved in 600 g water were added and the pH value was then adjusted to approximately 4-5 by addition of a 2M sodium hydroxide solution. The organic phase was separated, washed with brine, dried over sodium sulfate and concentrated in vacuo to afford rac 6-(4-chloropyridin-2-yl)-2-methyl-6-oxohexanoic acid (rac-Compound 10). Yield: 331 g (82%).

### Example 8: Recrystallization of the salt (R)-6-(4-chloropyridin-2-yl)-2-methyl-6-oxohexanoic acid (1R,2S)-erythro-2-amino-1,2-diphenylethanol H₂O obtained in previous step

407 g of the diastereomeric salt (R)-6-(4-chloropyridin-2-yl)-2-methyl-6-oxohexanoic acid·(1R,2S)-erythro-2-amino-1,2-diphenylethanol·H₂O (diastereomeric salt of Compound 10A) obtained in the previous step (Example 6) were recrystallized from 4.0 kg of ethanol (96% by volume) by dissolving the salt at a temperature of 70° C. and then slow cooling the solution to a temperature of 10° C. The obtained crystalline precipitate was filtered off, washed with cold ethanol (96% by volume) and dried to afford purified (R)-6-(4-chloropyridin-2-yl)-2-methyl-6-oxohexanoic acid·(1R,2S)-erythro-2-amino-1,2-diphenylethanol·H₂O. Yield: 326 g (80%). S/R ratio: 7.7:92.3.

### Example 9: Conversion of (R)-6-(4-chloropyridin-2-yl)-2-methyl-6-oxohexanoic acid (1R,2S)-erythro-2-amino-1,2-diphenylethanol H₂O into (R)-6-(4-chloropyridin-2-yl)-2-methyl-6-oxohexanoic acid

182 g of (R)-6-(4-chloropyridin-2-yl)-2-methyl-6-oxohexanoic acid·(1R,2S)-erythro-2-amino-1,2-diphenylethanol·H₂O was dissolved in 2.7 kg of TBME and 3.5 kg of a 0.33M aqueous solution of hydrochloric acid until two homogenous phases were formed. The organic phase was separated, dried over sodium sulfate and concentrated in vacuo to afford the free acid (R)-6-(4-chloropyridin-2-yl)-2-methyl-6-oxohexanoic acid (Compound 10A). Yield: 93.2 g (98%).

### Example 10: Conversion of (R)-6-(4-chloropyridin-2-yl)-6,6-dimethoxy-2-methylhexanoic acid to (R)-6-(4-chloropyridin-2-yl)-6,6-dimethoxy-2-methylhexanoic acid DCHA salt

Step (a

(R) 6-(4-chloropyridin-2-yl)-2-methyl-6-oxohexanoic acid (Compound 10A) (5.0 g) was dissolved in dry methanol (20 g) and cooled to 0° C. Trimethylsilyl chloride (3.1 mL) was added slowly, followed by trimethyl orthoformate (6.4 mL). The mixture was stirred at room temperature for 12 h and then at 40° C. for 12 h until conversion was complete. When the reaction was complete, the reaction mixture was cooled to 0° C. Barium hydroxide (9.5 g; the octahydrate can also be used) suspended in water (50 g) was added and the reaction stirred overnight at room temperature. When conversion was complete, saturated sodium dihydrogen phosphate solution was added until pH=4-5 is reached. The mixture was then extracted with TBME (2×20 g), the organic phase washed with brine (2×20 g), dried over sodium sulfate and the volatiles were removed in vacuo to give the product as a slightly yellow oil. Yield: 5.78 g (98%).

Step (b)

(R)-6-(4-chloropyridin-2-yl)-6,6-dimethoxy-2-methylhexanoic acid (Compound 11) (368 g) was dissolved in TBME (2400 g) and warmed to 40° C. Dicyclohexylamine (175 g) was slowly added, and after formation of precipitate stirring was continued for 15 min until the stirrability improved. The rest of the dicyclohexylamine (174 g) was then slowly added. The mixture was slowly cooled to room temperature and the solid was filtered off and washed with TBME to yield the product as white needles. Yield: 671.0 g with approx. 1.25 eq. DCHA and 14.5% TBME. 570 g product (88.5%)

### Example 11: Nitropyrazole Reduction

Step (a)

1-(Difluoromethyl)-4-nitro-pyrazole (Compound 12) (50 g), Pt/V/C (2.5 g) and THF (100 mL) were charged into hydrogenation reactor. After amortization procedure, the reactor was pressurized under hydrogen atmospheres (0.27-0.34 MPa) and stirred until complete conversion at 30°-35° C. under H₂ flow. After complete conversion, the organic layer was filtered to remove the catalyst and concentrate to dryness to afford 1-(difluoromethyl)-4-amino-pyrazole (Compound 13) in quantitative yield.

Step (b)

1-(Difluoromethyl)-4-amino-pyrazole (10 g) was dissolved in 2-propanol (80 mL) in presence of HCl in 2-propanol (22 mL) at 0°-5° C. The reaction mixture was slowly warmed to 25° C. over 30 minutes and heptane (100 mL) was charged. After 10 minutes stirring, the reaction mixture was filtered washed with heptane. The isolated material was dried under reduced pressure at 40° C. 7.9 g of 1-(difluoromethyl)-4-amino-pyrazole·HCl salt (HCl salt of Compound 13) was isolated in 60% yield.

### Example 12: Amide Coupling Step

In a 500 mL RBF reactor, (R)-6-(4-chloropyridin-2-yl)-6,6-dimethoxy-2-methylhexanoic acid DCHA salt (Compound 11 DHCA) (50 g) was suspended in n-butyronitrile (300 mL) and washed with acidic water (300 mL+H₃PO₄ 85 wt. %, 5.21 g) until a pH 5 was achieved. After phase separation, the organic layer was concentrated to 200 mL. In a second reaction, 1,1′-carbonyldiimidazole (1,2 equiv., 17.6 g) was suspended in n-butyronitrile (100 mL) and the temperature was increased to 40° C. The 200 mL solution containing (R)-6-(4-chloropyridin-2-yl)-6,6-dimethoxy-2-methylhexanoic acid were added dropwise to the CDI suspension over 2 hours. After complete conversion, 1-(difluoromethyl)-1H-pyrazol-4-amine. HCl salt (1.2 equiv., 18.4 g) was added in one portion and the reaction mixture was stirred for 6 hours at 40° C. After completion and cooling to 25° C., the organic layer was washed first with aqueous sodium hydroxide till pH 12, then aqueous phosphoric acid (300 mL, 5.21 g phosphoric acid in water) and a second time with aqueous phosphoric acid (300 mL, 0.521 g phosphoric acid in water) and finally with aqueous sodium hydroxide (300 mL, 1.45 g NaOH). The organic layer was concentrated till 200 mL containing 34 g of (R)-6-(4-chloropyridin-2-yl)-N-(1-(difluoromethyl)-1H-pyrazol-4-yl)-6,6-dimethoxy-2-methylhexanamide (Compound 14) corresponding to 90% yield.

### Example 13: Synthesis of (R)-2¹-(difluoromethyl)-9,9-dimethoxy-5-methyl-2¹H-3-aza-1(4,2)-pyridina-2(5,4)-pyrazolacyclononaphan-4-one-HCl

Step (a)

In a RBF with overhead stirrer, was charged K₂CO₃ (1.2 equiv., 12.05 g), n-butyronitrile (300 mL), Pd(allyl)(Catacxium A)Cl (0.01 equiv., 0.393 g) and pivalic acid (0.1 equiv., 0.822 mL). After amortisation under nitrogen atmosphere, the reaction mixture was heated to 115° C. and (R)-6-(4-chloropyridin-2-yl)-N-(1-(difluoromethyl)-1H-pyrazol-4-yl)-6,6-dimethoxy-2-methylhexanamide (Compound 14) (135.7 g) was added dropwise over 5 hours. The reaction mixture was kept at 115° C. until complete conversion. After cooling to room temperature, the reaction mixture was filtered over decalite (0.1 g/g, 100 mass %) and the cake was washed with butyronitrile to afford (R)-2¹-(difluoromethyl)-9,9-dimethoxy-5-methyl-2¹H-3-aza-1(4,2)-pyridina-2(5,4)-pyrazolacyclononaphan-4-one (Compound 15) in 90% yield.

Step (b)

To a 100 mL reactor with overhead stirrer, a butyronitrile solution containing of (R)-2¹-(difluoromethyl)-9,9-dimethoxy-5-methyl-2¹H-3-aza-1(4,2)-pyridina-2(5,4)-pyrazolacyclononaphan-4-one (Compound 15) (70 g with 4.089 wt. % assay) was stirred at 15° C. A solution of HCl in 2-propanol (2.06 mL, 1.0 equiv.·HCl) was added dropwise over 30 minutes and stirred for 2 extra hours at 15° C. The reaction mixture was filtered and washed with n-butyronitrile. The isolated material was dried under reduced pressure at 45° C. overnight. (R)-2¹-(difluoromethyl)-9,9-dimethoxy-5-methyl-2¹H-3-aza-1(4,2)-pyridina-2(5,4)-pyrazolacyclononaphan-4-one·HCl (HCl salt of Compound 15) (3 g) salt was isolated in 96% yield.

### Example 14: Conversion of (R)-21-(difluoromethyl)-9,9-dimethoxy-5-methyl-2¹H-3-aza-1(4,2)-pyridina-2(5,4)-pyrazolacyclononaphan-4-one-HC to (5R,9S)-amino-21-(difluormethyl)-5-methyl-2′H-3-aza-1(4,2,)pyridine-2(5,4)-pyrazolocyclononaphon-4-one

(R)-2¹-(difluoromethyl)-9,9-dimethoxy-5-methyl-2¹H-3-aza-1(4,2)-pyridina-2(5,4)-pyrazolacyclononaphan-4-one-HCl (10 g) was dissolved in water (75 mL) and the reaction mixture was heated for 18 hours. Under reduced pressure, 25% of the solvent was removed by distillation under reduced pressure and replaced with water. The reaction mixture was cooled to 5° C. and 2.35 eq. HCl (pH=0.98) were added followed by 4.05 eq. isopropylamine while temperature slowly rose to 20° C. Finally, 1 wt. % pyridoxal 5′-phosphate hydrate (5-PLP) and 1.1 wt. % transaminase CDX-050 (also known as transaminase ATA-486) were added and the reaction mixture was heated to 50° C. and stir for 24 hours. After complete conversion concentrated HCl was added until pH 7 was achieved. Diatomaceous earth (4 wt. %) was charged and the reaction mixture was heated to 80° C. for 2 hours. The suspension was cooled to 20° C. and filtered. To the obtained filtrate, was added dropwise NaOH 1M until pH 10 was achieved. After 2 hours stirring, the reaction mixture was filtered and the cake was washed twice with water to afford the desired product 83% yield (Compound C) (6.7 g).

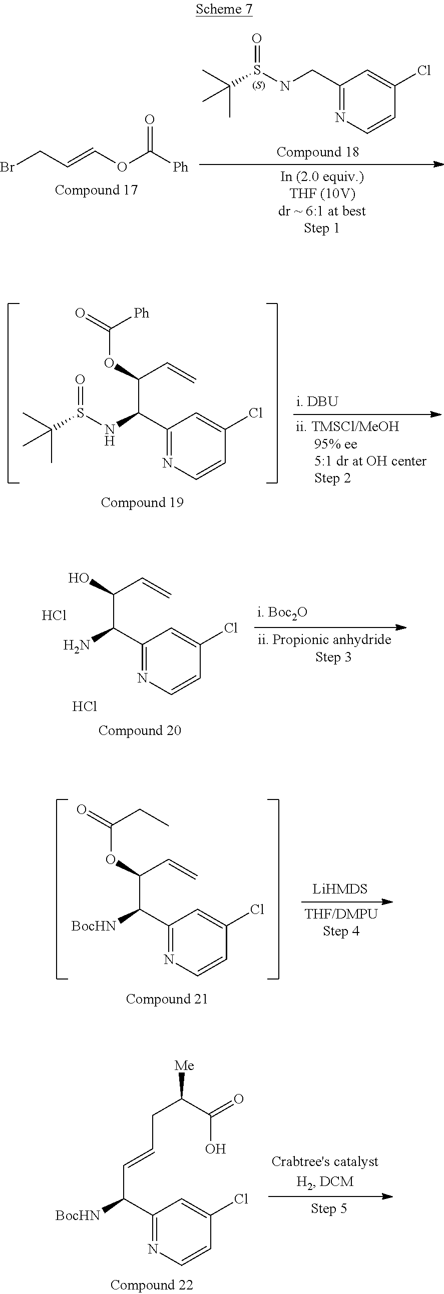

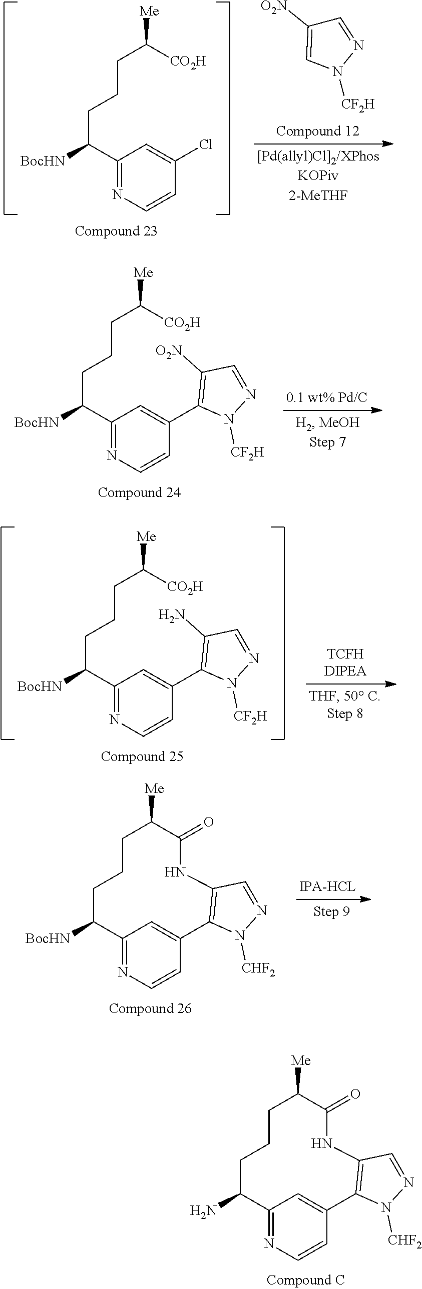

### Example 15: Synthesis of Compound (1R,2S)-1-amino-1-(4-chloropyridin-2-yl)but-3-en-2-ol bis hydrochloride salt

(E)-3-bromoprop-1-en-1-yl benzoate (Compound 17) (198 g, 821 mmol) was added to a reactor followed by THF (1 L) and indium (94.18 g, 821 mmol) at 20° C. The solution was allowed to stir at 20° C. for NLT 12 h. The solution was cooled to NMT 5° C. A solution of (S,E)-N-((4-chloropyridin-2-yl)methylene)-2-methylpropane-2-sulfinamide (Compound 18) (100 g, 408.5 mmol) in THF (300 mL) was added so as to maintain the temp of NMT 10° C. After the addition, the solution was allowed to stir at 10° C. for NLT 1 h. The solution was filtered and diluted with MTBE (500 mL). The organics were then washed with aq. citric Acid (200 g in 1 L water). This was repeated once. The organics were then washed with water (500 mL) twice followed by a sat. aq. NaHCO₃ (500 mL) wash. The organics were concentrated to 1-2 V, and then diluted with MeOH (10V). This was repeated until there was NMT 2% MTBE in MeOH. Once the MTBE was NMT 2%, the organics were adjusted to a total of 3V MeOH. DBU (81.7 mmol, 0.20 equiv.) was added to the solution. The solution was heated to 50° C. and was held for NLT 2 h at 50° C. Once complete by HPLC, TMSCl (2043 mmol, 5.0 equiv.) was added at 50° C. The solution was held at 50° C. for NMT 30 min. Once complete by HPLC, The solution was concentrated to 5V total. At 50° C., iPrOAc (3V) was added over NLT 1 h. The solution was then cooled to NMT 15° C. The solution was held at NMT 15° C. for NLT 2 h. The slurry was filtered to afford (1R,2S)-1-amino-1-(4-chloropyridin-2-yl)but-3-en-2-ol bis hydrochloride salt (Compound 20) (100 g, 90% yield) as a white solid. ¹H NMR (DMSO-d₆, 600 MHz): δ 8.81 (broad s, 1H), 8.57 (d, J=5.4 Hz, 1H), 7.77 (d, J=1.92 Hz, 1H), 7.58 (dd, J=1.92, 5.4 Hz, 1H), 5.85 (m, 1H), 5.17 (dt, J=1.42, 17.3 Hz, 1H), 5.11 (dt, J=1.42, 10.5 Hz, 1H), 4.60 (m, 1H), 4.49 (m, 1H). ¹³C NMR (DMSO-d₆, 151 MHz): δ 155.4, 149.4, 143.6, 136.3, 124.0, 123.9, 117.3, 71.5, 58.0. HRMS: ESI positive HRMS [M+H]⁺: C₉H₁₂N₂OCl, calc'd: 199.0633, observed: 199.0633.

### Example 16: Synthesis of compound (2R,6S,E)-6-((tert-butoxycarbonyl)amino)-6-(4-chloropyridin-2-yl)-2-methylhex-4-enoic acid (Compound 22)

Step 1

(1R,2S)-1-amino-1-(4-chloropyridin-2-yl)but-3-en-2-ol bis hydrochloride salt (5.5 g), toluene (55 mL), sat. NaHCO₃ (aq) (55 mL), and Boc₂O (4.6 g) were added to a round bottom flask with a stir bar. The reaction was allowed to stir at 20° C. for 4 h. The reaction was diluted with brine (30 mL), and the layers were separated.

Step 2

Propionic anhydride (3.5 g) was added to the organics, followed by DMAP (250 mg). The reaction was stirred for 2 h at room temperature. MeOH (3 mL) was added to quench the excess anhydride. After 30 min, the sample was washed with brine to remove DMAP, followed by 1:1 NaHCO₃:Brine, then brine. The toluene was distilled on rotovap (10 torr, 40 C), then 50 mL more toluene was added and the distillation was repeated to remove t-BuOH and water by azeotropic distillation, to yield a clear residue.

Step 3

The residue was dissolved in THF (100 mL) and DMPU (68 mL), and 1M TBSCl in THF (35 mL, 2.1 equiv.) was added. The reaction atmosphere was charged with an inert atmosphere of nitrogen (N₂), then the reaction was cooled to −78° C. 1.5M LiHMDS in THF (23 mL) was added dropwise. The reaction was aged at −78° C. for 30 min, then was allowed to warm to −10° C. over the course of about 1 h as the bath slowly warmed. The reaction was quenched with the addition of solid NH₄F (2.4 g), then water (20 mL). Water was then continually added until all of the solid NH₄F dissolved and a single homogeneous solution was observed (50 mL total water). Toluene (100 mL) was added followed by more water (100 mL) and saturated brine (20 mL). The aqueous phase was collected and organics were washed with sat NaHCO₃ (aq) (50 mL). The combined aqueous fractions were adjusted to pH=5 with phosphoric acid. The product was extracted into 1:1 toluene:MTBE (300 mL), then washed with 2:1 water:brine (200 mL). The volatiles were removed under reduced pressure to afford crude solid. The crude solid was dissolved in toluene (40 mL) at 70° C., then hexanes (40 mL) was added. The reaction was cooled to 55° C., and seed crystals of the product (50 mg) were added. The reaction was left stirring at 50° C. for 4 h. The sample was allowed to cool to room temperature over an hour and stir at room temperature for an additional hour. The crystals were filtered off and washed with 1:1 hexanes toluene (20 mL), then hexanes (20 mL). ¹H NMR (DMSO-d₆, 600 MHz): δ 8.46 (d, J=5.4 Hz, 1H), 7.48 (broad s, partially overlapped, 1H), 7.47 (d, J=1.92 Hz, 1H), 7.39 (dd, J=1.92, 5.4 Hz, 1H), 5.61 (m, 1H), 5.56 (m, 1H), 5.17 (m, 1H), 2.35 (m, 1H), 2.27 (m, 1H), 2.04 (m, 1H), 1.37 (s, 9H), 1.00 (d, J=6.9 Hz, 3H). ¹³C NMR (DMSO-d₆, 151 MHz): δ 176.7, 163.5, 154.8, 150.3, 143.3, 130.9, 129.0, 122.2, 120.8, 78.2, 57.4, 38.4, 35.5, 28.1, 16.3. HRMS (C₁₇H₂₄O₄N₂Cl): Theo. Mass 355.1419, Delta (ppm) −0.43, RDP equiv. 6.5 Found: m/z 355.1418.

### Example 17: Synthesis compound tert-butyl ((5R,9S)-21-(difluoromethyl)-5-methyl-4-oxo-2¹H-3-aza-1(4,2)-pyridina-2(5,4)-pyrazolacyclononaphane-9-yl)carbamate (Compound 26)

In a 40 mL vial containing (2R,6S,E)-6-((tert-butoxycarbonyl)amino)-6-(4-chloropyridin-2-yl)-2-methylhex-4-enoic acid (Compound 22) (226.1 mg) was added Crabtree's catalyst (12.7 mg) and DCM (4.5 mL). About 2.25 mL of that solution was subjected to about 10 nitrogen cycles, followed by 10 H₂ cycles under stirring. 40 psi hydrogen pressure was applied and the mixture was shaken at 500 rpm for 2 days. The resulting slurry was filtered and the wet cake (2R,6S)-6-((tert-butoxycarbonyl)amino)-6-(4-chloropyridin-2-yl)-2-methylhexanoic acid (Compound 23) was further wash with n-heptane.

In a 4 mL vial, was added allyl palladium (II) chloride dimer (3.5 mg), X-Phos (10.2 mg) and 2-Me THF (2.8 mL). After 30 min, a solution was obtained. Into a 8 mL vial was added (2R,6S)-6-((tert-butoxycarbonyl)amino)-6-(4-chloropyridin-2-yl)-2-methylhexanoic acid (Compound 23) (67.2 mg), 1-(difluoromethyl)-4-nitro-1H-pyrazole (Compound 12) (41.4 mg) and potassium trimethylacetate (114.8 mg). To this vial was added the Pd/Xphos solution (1.34 mL and the reaction mixture was heated to 80° C. with stirring. At this point the reaction mass is a white slurry. After 17 h, the orange brown slurry was cooled to 20° C. A kicker charge of potassium trimethylacetate (54.9 mg) was added and reaction mass was heated back to 80° C. for 23 h. After cooling down to 20° C., 0.1 mL of a solution of allyl palladium (II) chloride dimer (3.5 mg), X-Phos (10.2 mg) and 2-Me THF (0.2 mL). After another heating to 80° C. for 18 h, the reaction was deemed complete and the brown slurry was cooled to 20° C. The organic phase was washed with 20 wt. % K₃PO₄ and the phases were separated. The organic phase was extracted again with 20 wt. % K₃PO₄ and the phases were separated. To the combined aqueous phase was added 2-MeTHF and darco and a 20 wt. % aqueous citric acid until pH 4 was achieved. The biphasic reaction mass was filtered through a filter and the phases were separated. The organic top layer was washed with aq NaCl and then was dried over MgSO₄, filtered and concentrated. The oily residue was purified by column chromatography (SiO₂) using a gradient of 20 to 100% acetone in hexane to isolate (2R,6S)-6-((tert-butoxycarbonyl)amino)-6-(4-(1-(difluoromethyl)-4-nitro-1H-pyrazol-5-yl)pyridin-2-yl)-2-methylhexanoic acid (Compound 24). LC/MS showed a mass of M+1 484.15.

(2R,6S)-6-((tert-butoxycarbonyl)amino)-6-(4-(1-(difluoromethyl)-4-nitro-1H-pyrazol-5-yl)pyridin-2-yl)-2-methylhexanoic acid (Compound 24) (43.7 mg) was then dissolved in 2-MeTHF (0.43 mL). After additional 0.43 mL of 2-MeTHF, Pd/C (5.1 mg) was added. The reaction mass was subjected to hydrogenation at 25 psi H₂ pressure and shaked for 18 h. The reaction mass was then filtered and the solid was rinse with DCM. The filtrate was then concentrated to dryness.

The resulting (2R,6S)-6-(4-(4-amino-1-(difluoromethyl)-1H-pyrazol-5-yl)pyridin-2-yl)-6-((tert-butoxycarbonyl)amino)-2-methylhexanoic acid (Compound 25) was then subjected to the macrolactamization as follow: (2R,6S)-6-(4-(4-amino-1-(difluoromethyl)-1H-pyrazol-5-yl)pyridin-2-yl)-6-((tert-butoxycarbonyl)amino)-2-methylhexanoic acid (100 mg) in THF (2.0 mL) was added over 2 h by a syringe pump into a solution of TCFH (0.158 g) and DIPEA (0.135 mL) in THF (15 mL). After 0.5 h, the reaction mixture was concentrated to dryness and was redissolved in EtOAc (15 mL). The resulting mixture was washed with 15% aq. NH₄Cl. after phase separation, the organic phase was dried over MgSO₄, filtered and concentrated. The residue was purified by ISCO (30-90% EtOAc/Heptane) and after solvent evaporation of the fractions, 85 mg of tert-butyl ((5R,9S)-2¹-(difluoromethyl)-5-methyl-4-oxo-2¹H-3-aza-1(4,2)-pyridina-2(5,4)-pyrazolacyclononaphane-9-yl)carbamate (Compound 26) was obtained. ¹H NMR (400 MHz, DMSO-d₆) 9.32 (s, 1H), 8.71 (d, J=5.0 Hz, 1H), 7.96 (t, J=58 Hz, 1H), 7.43 (s, 1H), 7.32 (d, J=4.8 Hz, 1H), 7.22 (d, J=7.3 Hz, 1H), 4.66 (d, J=8.3 Hz, 1H), 2.62 (br.s., 1H), 1.88 (d, J=12.8 Hz, 1H), 1.77-1.59 (m, 2H), 1.42-1.28 (m, 9H), 1.15 (d, J=18.2 Hz, 2H), 0.83 (d, J=7.0 Hz, 3H). MS(ESI) m/z: 436.3 [M+H]⁺.

### Example 18: Synthesis of (5R,9S)-9-amino-21-(difluoromethyl)-5-methyl-2′H-3-aza-1(4,2)-pyridina-2(5,4)-pyrazolacyclononaphan-4-one (Compound C)

In a clean reactor, was charged MeOH (24.2 Kg), tert-butyl ((5R,9S)-2¹-(difluoromethyl)-5-methyl-4-oxo-2¹H-3-aza-1(4,2)-pyridina-2(5,4)-pyrazolacyclononaphane-9-yl)carbamate (Compound 26) (3 kg) and 6N IPA/HCl (6.2 L). The temperature was raised to 45-55° C. for at least 2 h or until the reaction is deemed complete. The reaction mass was then concentrated down to 9 L at 55° C. under 450 mmHg. Then MeOH (48.2 Kg) was charged and the reaction mass was concentrated down to 9 L. The above was repeated another time. Dowex monoshere Monosphere 550A (OH), was washed 3 time with dry methanol (48 kg each) until KF was <3.0 wt. %. To the methanolic solution of (5R,9S)-9-amino-2¹-(difluoromethyl)-5-methyl-2¹H-3-aza-1(4,2)-pyridina-2(5,4)-pyrazolacyclononaphan-4-one was then added methanol (4 L), and dried dowex Dowex resin and stir until pH was >8.5. The reaction mass was filtered through celite. The reactor and celite were washed with methanol (6.0 L and 50.70 kg) respectively.

The combined stream was concentrated to 15 L. At this point ACN (47.3 kg) was added and the reaction mass was concentrated down to 30 L. The operation is repeated 3 more times. Once the reaction solution showed a KF<0.5 wt. %, the temperature was dropped to 20° C. After 1 h aging the resulting slurry was filtered and the wet cake washed with ACN (9 L). The wet cake was dried at 80° C. for at least 6 h until the compound was deemed dried. (5R,9S)-9-amino-2¹-(difluoromethyl)-5-methyl-2¹H-3-aza-1(4,2)-pyridina-2(5,4)-pyrazolacyclononaphan-4-one (Compound C) was obtained in 76.3% yield or 1.57 kg.

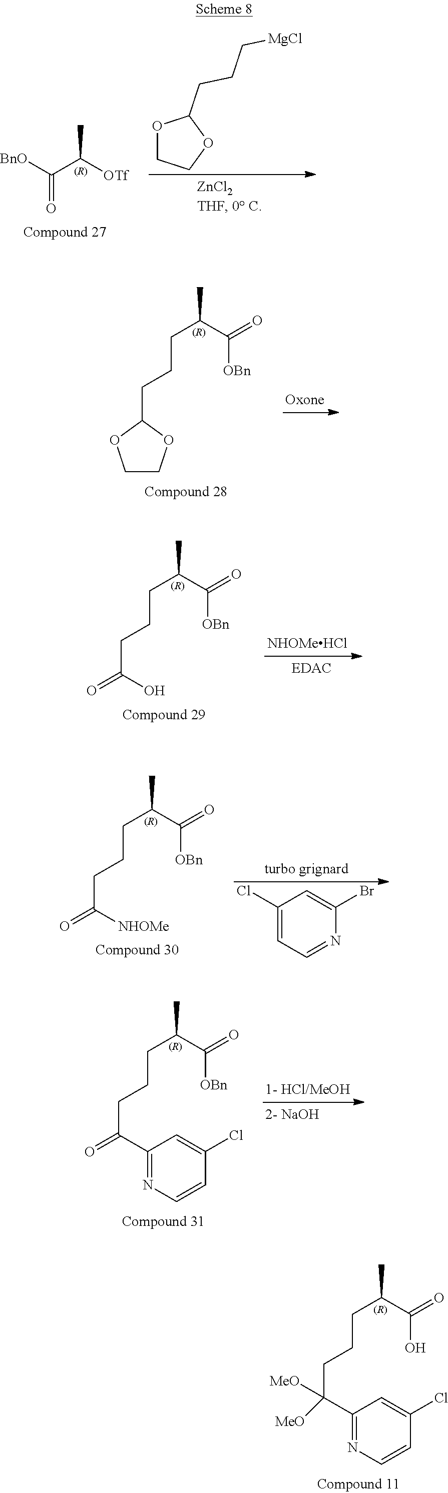

### Synthesis of (R)-6-(4-chloropyridin-2-yl)-6,6-dimethoxy-2-methylhexanoic acid via Lactic Acid

### Example 19: Synthesis of benzyl (R)-5-(1,3-dioxolan-2-yl)-2-methylpentanoate (Compound 32)

The following were added to a flask: THF (3.75 mL), ZnCl₂ (0.5 M in THF, 1 mL). The solution was cooled to 0° C. The following was added over 5 min: chloro-[3-(1,3-dioxolan-2-yl)propyl]magnesium (4.80 mmol, 8.0 mL) and benzyl (2R)-2-(trifluoromethylsulfonyloxy)propanoate (3.20 mmol, 1.00 g). The solution was then allowed to stir at 0° C. for NLT 12 h. The solution was then diluted with sat. aq. NH₄Cl (5V) and was extracted with MTBE (10V). The organics were concentrated to a residue and was purified by iSCO [1% to 40% EtOAc in Hexane] to afford benzyl (2R)-5-(1,3-dioxolan-2-yl)-2-methylpentanoate (Compound 28) (39% Yield, 0.351 g, 0.351 g) as an oil. 1H NMR: ¹H NMR (500 MHz, CHLOROFORM-d) δ 7.42-7.32 (m, 5H), 5.14 (s, 2H), 4.87-4.80 (m, 1H), 4.00-3.82 (m, 4H), 2.57-2.48 (m, 1H), 1.81-1.70 (m, 1H), 1.70-1.62 (m, 2H), 1.55-1.40 (m, 3H), 1.20 (d, J=7.0 Hz, 3H). 13C NMR: ¹³C NMR (126 MHz, CDCl₃) δ 176.5, 136.2, 128.5, 128.1, 128.0, 104.3, 66.0, 64.8, 39.5, 33.7, 33.6, 21.7, 17.0. HRMS. ESI positive HRMS [M+H]⁺ C₁₆H₂₃O₄. Theoretical: 279.1591 [M+H]. Found: 279.1588 [M+H]

### Example 20: Synthesis of (R)-6-(benzyloxy)-5-methyl-6-oxohexanoic acid (Compound 29)

Benzyl (R)-5-(1,3-dioxolan-2-yl)-2-methylpentanoate (Compound 28) (10 g, 36 mmol) was dissolved in THF (100 mL) in a round bottom flask. Water (100 mL) and oxone (22.3 g) were added. The mixture was stirred at rt for 48 h. Heptane (50 mL) was added and the aqueous phase was separated. The organic phase was washed with water (50 mL), then brine (50 mL). The organics were concentrated by use of a rotovap to a residue. The residue was chromatographed on silica with 100% DCM-20% acetone in DCM. (R)-6-(benzyloxy)-5-methyl-6-oxohexanoic acid (Compound 29) was obtained as an oil (8.8 g, 97% yield). ¹H NMR (DMSO-d₆, 600 MHz): δ 7.31-7.39 (m, 5H), 5.10 (s, 2H), 2.49 (m, 1H), 2.19 (t, J=7.1 Hz, 2H), 1.60 (m, 1H), 1.48 (m, 2H), 1.41 (m, 1H), 1.09 (d, J=7.1 Hz, 3H). ¹³C NMR (DMSO-d₆, 151 MHz): δ 175.5, 174.3, 136.4, 128.5, 128.0, 127.8, 65.4, 38.6, 33.5, 32.7, 22.2, 16.8. HRMS data: C₁₄H₁₉O₄[M+H]⁺ expected: 251.1278 found: 251.1286.

| | Theo. | Delta | RDB | |
|---|---|---|---|---|
| m/z | Mass | (ppm) | equiv. | Composition |
| 251.1286 | 251.1278 | 3.12 | 5.5 | C₁₄H₁₉O₄ [M + H]⁺ |
| 273.1106 | 273.1097 | 3.18 | 5.5 | C₁₄H₁₈O₄Na [M + Na]⁺ |

### Example 21: Synthesis of benzyl (R)-6-(methoxy(methyl)amino)-2-methyl-6-oxohexanoate (Compound 30)

(R)-6-(benzyloxy)-5-methyl-6-oxohexanoic acid (1 g), N,O-dimethylhydroxylamine hydrochloride (470 mg), triethylamine (1.7 mL), 1-(3-dimethylaminopropyl)-3-ethylcarbodiimide hydrochloride (930 mg), and dichloromethane (10 mL) were placed in a round bottom flask with a stir bar. The reaction was stirred at rt for 16 h. The organic phase was placed in a separatory funnel and washed with water, then 1N HCl (aq), then NaHCO₃ (aq). The organics were then dried over MgSO₄, filtered, and concentrated to a residue by use of a rotovap. The residue was chromatographed with 100% DCM-10% MTBE in DCM. Benzyl (R)-6-(methoxy(methyl)amino)-2-methyl-6-oxohexanoate was obtained in 57% yield (Compound 30) (665 mg). ¹H NMR (DMSO-d₆, 600 MHz): δ 7.28-7.35 (m, 5H), 5.05 (s, 2H), 3.57 (s, 3H), 3.03 (s, 3H), 2.45 (m, 1H), 2.30 (bs, 2H), 1.55 (m, 1H), 1.44 (m, 2H), 1.37 (m, 1H), 1.05 (d, J=7.1 Hz, 3H). ¹³C NMR (DMSO-d₆, 151 MHz): δ 176.3, 173.8, 136.7, 129.0, 128.5, 128.2, 65.9, 61.4, 39.1, 33.2, 32.1, 31.3, 22.2, 17.2. HRMS data: C₁₆H₂₄O₄N [M+H]⁺ expected: 294.1700, found: 294.1708

| | Theo. | Delta | RDB | |
|---|---|---|---|---|
| m/z | Mass | (ppm) | equiv. | Composition |
| 294.1708 | 294.1700 | 2.84 | 5.5 | C₁₆H₂₄O₄N[M + H]⁺ |
| 316.1527 | 316.1519 | 2.41 | 5.5 | C₁₆H₂₃O₄NNa[M + Na]⁺ |

### Example 22: Synthesis of benzyl (R)-6-(4-chloropyridin-2-yl)-2-methyl-6-oxohexanoate (Compound 31)

2-bromo-4-chloropyridine (240 mg) and THF (3 mL) were placed in a round bottom flask with a stir bar under an inert atmosphere of nitrogen. 1.3M turbo Grignard in THF (870 μl) was added to the flask at room temperature. The reaction was stirred at room temperature for 1 h. The reaction was then cooled to −78° C., and benzyl (R)-6-(methoxy(methyl)amino)-2-methyl-6-oxohexanoate (300 mg) dissolved in THF (1 mL) was added via syringe. After 10 minutes, the flask was removed from the cooling bath and allowed to warm to room temperature before being quenched with NaHCO₃(aq) and diluted with EtOAc. The organic phase was placed in a separatory funnel and the phases were separated. The organics were then dried over MgSO₄, filtered, and concentrated to a residue by use of a rotovap. The residue was chromatographed with 1:9 EtOAc:heptane. Benzyl (R)-6-(4-chloropyridin-2-yl)-2-methyl-6-oxohexanoate (Compound 31) was obtained as a clear oil (170 mg, 48% yield). ¹H NMR (DMSO-d₆, 600 MHz): δ 8.68 (broad d, J=5.4 Hz, 1H), 7.92 (m, 1H), 7.79 (m, 1H), 7.29-7.36 (m, 5H), 5.09 (s, 2H), 3.13 (t, J=7.4 Hz, 2H), 2.52 (m, 1H), 1.62 (m, 3H), 1.46 (m, 1H), 1.10 (d, J=7.1 Hz, 3H). ¹³C NMR (DMSO-d₆, 151 MHz): δ 199.8, 175.4, 154.1, 150.7, 144.3, 136.3, 128.4, 127.9, 127.7, 127.4, 121.2, 65.3, 38.6, 36.9, 32.7, 20.9, 16.7.

HRMS data:

| | Theo. | Delta | RDB | |
|---|---|---|---|---|
| m/z | Mass | (ppm) | equiv. | Composition |
| 346.1212 | 346.1204 | 2.14 | 9.5 | C₁₉H₂₁O₃NCl [M + H]⁺ |
| 368.1030 | 368.1024 | 1.54 | 9.5 | C₁₉H₂₀O₃NClNa [M + Na]⁺ |

### Example 23: Synthesis of (R)-6-(4-chloropyridin-2-yl)-6,6-dimethoxy-2-methylhexanoic acid (Compound 32)

Benzyl (R)-6-(4-chloropyridin-2-yl)-2-methyl-6-oxohexanoate (30 mg) was dissolved in MeOH (300 μl). TMSCl (20 μl) and TMOF (100 μl) were added and the reaction was maintained at rt for 12 h. The reaction was concentrated to a residue by use of a rotovap. 1N NaOH (aq) (1 mL) and MeOH (1 mL) were added to the residue, and the reaction was maintained at room temperature for 1 h. The reaction was placed into a separatory funnel, and the aqueous phase was washed with hexanes. The aqueous phase was then acidified with 1N HCl (aq) to pH 3 and then extracted with EtOAc. The organics were dried over MgSO₄, filtered, and concentrated to a residue by use of a rotovap. The product was isolated in 77% yield (22 mg). NMR matches authentic product, and the ee is measured to be 94% by chiral HPLC.

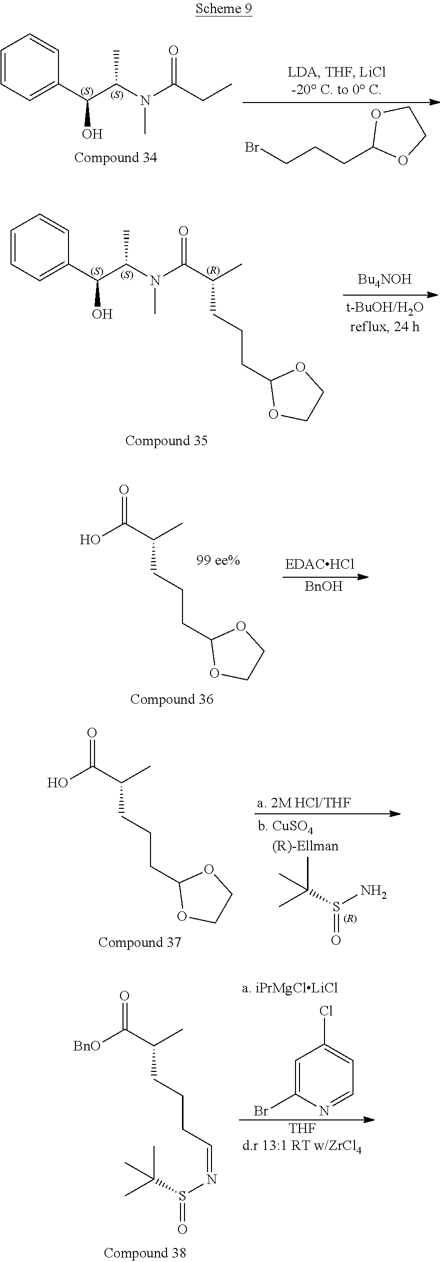

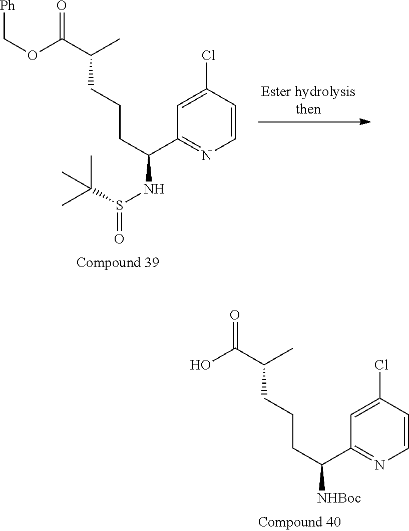 [SMILES: C[C@H](CCC[C@H](N[S@](=O)C(C)(C)C)c1cc(Cl)ccn1)C(=O)OCc1ccccc1 ; C[C@H](CCC[C@H](NC(=O)OC(C)(C)C)c1cc(Cl)ccn1)C(=O)O]

### Synthesis of (2R,6S)-6-(tert-butoxycarbonyl)amino-6-(4-chlorpyridin-2yl)-2-methyl hexanoic acid via Pseudoephedrine (Compound 40)

### Example 24: Synthesis of (2R)-5-(1,3-dioxolan-2-yl)-2-methyl-pentanoic acid (Compound 36)

Lithium Chloride (2.1 g, 51 mmol) was added to a flask. The flask was placed under vacuum (˜25 mmHg) and was heated with a heat gun (+100° C., NLT 30 min) to remove any traces of water. The flask was cooled to 20° C. and THF (22 mL) was added followed by diisopropylamine (5.9 g, 58 mmol). The solution was cooled to −20° C. n-BuLi [2.5 M in Hexanes] (23 mL) was added over NLT 1 h. A solution of N-[(1S,2S)-2-hydroxy-1-methyl-2-phenyl-ethyl]-N-methyl-propanamide (Compound 34) (25 mmol, 5.6 g) in THF (11 mL) was added dropwise so as to maintain the temp NMT −10° C. The solution was then cooled to −20° C. and held for NLT 1 h. It was then warmed to 0° C. and held for NLT 3 h. The solution was then warmed to the 20° C. and held for NLT 45 min. The solution was then recooled to 0° C. and 2-(3-bromopropyl)-1,3-dioxolane (50.61 mmol, 10.39 g) was added dropwise. The solution was then allowed to warm to 20° C. in NLT 10 h. The reaction was quenched by the addition of sat. aq. NH₄Cl (5V) and followed by water (5V). MTBE (10V) was added and the aqueous phase was separated and removed. The organics were washed with Brine (5V). The organics were then concentrated to residue. The residue was dissolved into t-BuOH (78 mL) and Bu₄NOH (40 wt. % in water) (82 mL, 125 mmol) and water (233 mmol). The biphasic mixture was heated to reflux for NLT 24 h. The solution was cooled to 20° C. MTBE (5V) was added and the aqueous phase was separated. Discarded the organic phase. The aqueous layer was acidified to pH 5.0 using H₃PO₄. Once at pH 5.0, the aqueous phase was extracted with MTBE (5V, 3×). The combined organics were dried (Na₂SO₄), filtered and concentrated to afford crude (2R)-5-(1,3-dioxolan-2-yl)-2-methyl-pentanoic acid (Compound 36) (96% Yield, 4.52 g) as an oil. ¹H NMR (500 MHz, CHCl₃-d) δ 4.87 (t, J=4.7 Hz, 1H), 4.03-3.80 (m, 4H), 2.58-2.41 (m, 1H), 1.81-1.64 (m, 4H), 1.56-1.40 (m, 4H), 1.19-1.19 (m, 1H), 1.20 (d, J=7.0 Hz, 3H). ¹³C NMR (126 MHz, CDCl₃) δ 182.3, 104.3, 64.8, 39.3, 33.7, 33.3, 21.6, 16.8. HRMS: ESI positive HRMS [M−18+H]⁺ C₉H₁₅O₃ [M−18+H] Theoretical: 171.1016. Found: 171.1014

### Example 25: Synthesis of benzyl (2R)-5-(1,3-dioxolan-2-yl)-2-methyl-pentanoate (Compound 37)

The following were added to a flask: (2R)-5-(1,3-dioxolan-2-yl)-2-methyl-pentanoic acid (106.3 mmol, 20.00 g), dichloromethane (6631 mmol, 563.2 g, 425.0 mL), benzyl alcohol (127.5 mmol, 13.79 g, 13.20 mL), 4-dimethylaminopyridine (10.63 mmol, 1.298 g). The solution was cooled to 0° C. and the following was added: 1-(3-dimethylaminopropyl)-3-ethylcarbodiimide hydrochloride (117.9 mmol, 22.61 g). The solution was allowed to stir at 0° C. for 2 h and then warmed to 20° C. and held overnight. The solution washed with the following: sat. aq. NaHCO₃ (5V), water (5V), and Brine (5v). The organics were concentrated and purified by ISCO [0% to 40% EtOAc] to afford benzyl (2R)-5-(1,3-dioxolan-2-yl)-2-methyl-pentanoate (Compound 37) (90.0% Yield, 4.10 g, 4.10 g) as a clear oil. ¹H NMR (500 MHz, CHLOROFORM-d) δ 7.42-7.32 (m, 5H), 5.14 (s, 2H), 4.87-4.80 (m, 1H), 4.00-3.82 (m, 4H), 2.57-2.48 (m, 1H), 1.81-1.70 (m, 1H), 1.70-1.62 (m, 2H), 1.55-1.40 (m, 3H), 1.20 (d, J=7.0 Hz, 3H). ¹³C NMR (126 MHz, CDCl₃) δ 176.5, 136.2, 128.5, 128.1, 128.0, 104.3, 66.0, 64.8, 39.5, 33.7, 33.6, 21.7, 17.0. HRMS ESI positive HRMS [M+H]⁺ C₁₆H₂₃O₄ Theoretical: 279.1591 [M+H]. Found: 279.1588 [M+H].

### Example 26: Synthesis of benzyl (R,E)-6-(((R)-tert-butylsulfinyl)imino)-2-methylhexanoate (Compound 38)

¹H NMR (500 MHz, CDCl₃) δ 8.12-7.97 (m, 1H), 7.41-7.31 (m, 6H), 5.16-5.10 (m, 2H), 2.57-2.47 (m, 3H), 1.84-1.71 (m, 1H), 1.71-1.60 (m, 2H), 1.58-1.46 (m, 1H), 1.19 (s, 12H). ¹³C NMR (126 MHz, CDCl₃) δ 176.1, 169.0, 136.1, 128.6, 128.2, 66.1, 56.50, 39.3, 35.9, 33.2, 23.0, 22.3, 17.0. HRMS: ESI positive HRMS [M+H]⁺ C₁₈H₂₈O₃NS [M+H] Theoretical: 338.1784 [M+H]. Found: 338.1784 [M+H]

### Example 27: Synthesis of benzyl (2R,6S)-6-(((R)-tert-butylsulfinyl)amino)-6-(4-chloropyridin-2-yl)-2-methylhexanoate (Compound 39)

2-bromo-4-chloropyridine (350 mg) and THF (2.5 mL) were placed in a round bottom flask with a stir bar under an inert atmosphere of nitrogen. 1.3M turbo Grignard in THF (1.4 mL) was added to the flask at room temperature. The reaction was stirred at room temperature for 30 min. The reaction was then cooled to −78° C., and benzyl (R,E)-6-(((R)-tert-butylsulfinyl)imino)-2-methylhexanoate (500 mg) dissolved in THF (2 mL) was added via syringe. The reaction was allowed to warm to room temperature over the course of an hour before being quenched with NaHCO₃ (aq) and diluted with EtOAc. The organic phase was placed in a separatory funnel and the phases were separated. The organics were then dried over MgSO₄, filtered, and the volatiles were removed under reduced pressure to afford a concentrated residue. The residue was chromatographed with 1:1 EtOAc:hexanes-100% EtOAc and benzyl (2R,6S)-6-(((R)-tert-butylsulfinyl)amino)-6-(4-chloropyridin-2-yl)-2-methylhexanoate (Compound 39) was obtained as a clear oil (530 mg, 79% yield). ¹H NMR (DMSO-d₆, 600 MHz): δ 8.46 (d, J=5.4 Hz, 1H), 7.56 (d, J=1.8 Hz, 1H), 7.40 (dd, J=1.8, 5.4 Hz, 1H), 7.31-7.38 (m, 5H), 5.54 (d, J=6.8 Hz, 1H), 5.06 (s, 2H), 4.28 (m, 2H), 2.45 (m, 1H), 1.82 (m, 2H), 1.58 (m, 1H), 1.38 (m, 2H), 1.07 (s, 9H), 1.05 (d, J=7.1 Hz, 3H). ¹³C NMR (DMSO-d₆, 151 MHz): δ 175.5, 164.9, 150.1, 143.2, 136.3, 128.4, 127.9, 127.7, 122.3, 121.4, 65.3, 61.2, 55.3, 38.5, 36.1, 32.8, 23.0, 22.5, 16.7.

HRMS data:

| | Theo. | Delta | RDB | |
|---|---|---|---|---|
| m/z | Mass | (ppm) | equiv. | Composition |
| 451.1825 | 451.1817 | 1.93 | 8.5 | C₂₃H₃₂O₃N₂ClS [M + H]⁺ |

### Example 28: Synthesis of (2R,6S)-6-((tert-butoxycarbonyl)amino)-6-(4-chloropyridin-2-yl)-2-methylhexanoic acid (Compound 40)

Step 1

Benzyl (2R,6S)-6-(((R)-tert-butylsulfinyl)amino)-6-(4-chloropyridin-2-yl)-2-methylhexanoate (300 mg) was dissolved in MeOH (4 mL) in a round bottom flask, and 2N NaOH(aq) (1 mL) was added. The reaction was stirred at room temperature until the starting material was consumed. The reaction was diluted with water and washed with hexanes to remove benzyl alcohol. The mixture was acidified to pH 3 with citric acid, and the product was extracted into MTBE. The organics were concentrated to a residue by use of a rotovap.

Step 2

The residue was dissolved in MeOH (2.5 mL) in a round bottom flask, and 3 equiv. of TMSCl (250 μL) was added. After starting material was consumed, the volatiles were removed under vacuum.

Step 3

The residue was dissolved in THF (2.5 mL) and 1.5 equiv. Boc₂O (220 mg) and 3 equiv. triethylamine (280 μl) were added. After the intermediate was consumed, the reaction was diluted with 1N NaOH (10 mL) and hexanes (10 mL), and the organic layer was separated from the aqueous. The aqueous phase was acidified to pH 3 with saturated aqueous citric acid, and a white solid precipitated. The white solid was filtered off and washed with water (10 mL), then dried under stream of air overnight. (2R,6S)-6-((tert-butoxycarbonyl)amino)-6-(4-chloropyridin-2-yl)-2-methylhexanoic acid was obtained in 86% yield (203 mg). (Characterization matched that which was previously recorded for (2R,6S)-6-((tert-butoxycarbonyl)amino)-6-(4-chloropyridin-2-yl)-2-methylhexanoic acid.)

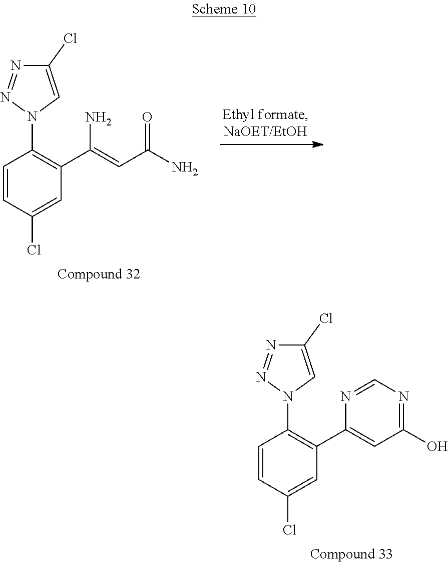 [SMILES: NC(=O)/C=C(\N)c1cc(Cl)ccc1-n1cc(Cl)nn1 ; Oc1cc(-c2cc(Cl)ccc2-n2cc(Cl)nn2)ncn1]

### Example 29: Synthesis of 6-(5-chloro-2-(4-chloro-1H-1,2,3-triazol-1-yl)phenyl)pyrimidin-4-ol (Compound 32)

In a 4 mL vial with red pressure-release cap, (Z)-3-amino-3-[5-chloro-2-(4-chlorotriazol-1-yl)phenyl]prop-2-enamide (Compound 32) (50 mg, 0.15262 mmol, 91 mass %, 1.0 equiv.) was weighed. To the content of the vial, ethyl formate (0.25 mL, 3.1 mmol, 100 mass %, 20 equiv.) was added followed by sodium ethoxide (21 wt. %) in ethanol (0.15 mL, 0.40 mmol, 21 mass %, 2.6 equiv.). The reaction mixture was heated to 55° C. for 2 h. This was followed by addition of HCl (1 mol/L) in deionized water (0.23 mL, 0.23 mmol, 1 mol/L, 1.5), biphasic. 2-methyltetrahydrofuran (0.5 mL, 5 mmol, 100 mass %, 30), shaken, split phase. The organic layer was washed with brine (0.5 mL) and split phase, followed by further drying of organic layer with Na₂SO₄, concentrated to ca. 5 V, charged 10 V of heptane, filtered, dried (80% yield). ¹H NMR (400 MHz, DMSO-d₆) δ 12.64 (br s, 1H), 8.75 (s, 1H), 8.06 (d, J=1.0 Hz, 1H), 7.88 (d, J=2.3 Hz, 1H), 7.85-7.71 (m, 2H), 6.34 (d, J=1.0 Hz, 1H).

### Example 30: XRPD Data for Crystalline Acetone Solvate Form of Compound (I)

TABLE 1
Peak list (higher than 5%) relative intensity
Pos. [°2Th.]

8.2321
10.0872
14.3163
16.1898
16.5524
17.5729
18.6786
19.1386
19.4389
19.5888
20.0236
21.2896
21.5821
22.0945
22.4947
23.5085
23.8930
24.9851
25.0767
25.4275
25.7772
26.4620
26.6794
27.1315
27.3484
28.9275
29.8608
30.2353
30.5195
30.7433
31.1200
31.5951
31.9657
32.7989
33.5411
34.0682
34.3433
34.6898
35.2079
35.6653
36.4135
36.7245
38.4035
38.9401
40.1133
43.2735
43.4015
43.7011
44.9886
46.1717
48.4294
49.2681

Procedure: X-ray power diffraction (XRPD) analysis was carried out on a PANalytical (Philips) X'PertPRO MPD diffractometer. The instrument is equipped with a Cl LFF X-ray tube. The compound was filled in a 16 mm cavity holder.

| Raw Data Origin | XRD measurement (*.XRDML) |
|---|---|
| Scan Axis | Gonio |
| Start Position [°2Th.] | 3.0084 |
| End Position [°2Th.] | 49.9794 |
| Step Size [°2Th.] | 0.0170 |
| Scan Step Time [s] | 60.7266 |
| Scan Type | Continuous |
| PSD Mode | Scanning |
| PSD Length [°2Th.] | 2.12 |
| Offset [°2Th.] | 0.0000 |
| Divergence Slit Type | Automatic |
| Irradiated Length [mm] | 10.00 |
| Specimen Length [mm] | 10.00 |
| Measurement Temperature [° C.] | 25.00 |
| Anode Material | Cu |
| Generator Settings | 40 mA, 45 kV |
| Goniometer Radius [mm] | 240.00 |
| Dist. Focus-Diverg. Slit [mm] | 100.00 |
| Incident Beam Monochromator | No |
| Spinning | Yes |

### Example 31: Infrared Data for Crystalline Acetone Solvate Form of Compound (I)

TABLE 2
IR data
Major IR bands (cm⁻¹)

| | | |
|---|---|---|
| 1709 (m) | 1676 (vs) | 1532 (m) |
| 1485 (m) | 1457 (m) | 1441 (m) |
| 1432 (m) | 1370 (m) | 1291 (m) |
| 1219 (m) | 1189 (m) | 1135 (m) |
| 1119 (m) | 1068 (m) | 1039 (m) |
| 994 (m) | 942 (m) | 883 (m) |
| 827 (s) | 801 (m) | 696 (m) |

(vs = very strong, s = strong, m = medium intensity)

Procedure: Micro Attenuated Total Reflectance (microATR) was used and the sample was analyzed using a suitable microATR accessory and the following measurement conditions:
apparatus: Thermo Nexus 670 FTIR spectrometer
number of scans: 32
resolution: 1 cm-1
wavelength range: 4000 to 400 cm-1
detector: DTGS with KBr windows
beamsplitter: Ge on KBr
micro ATR accessory: Harrick Split Pea with Si crystal

The examples and embodiments described herein are for illustrative purposes only and in some embodiments, various modifications or changes are to be included within the purview of disclosure and scope of the appended claims.

What is claimed is:

1. A process for preparing a crystalline solvate form of a compound represented by: 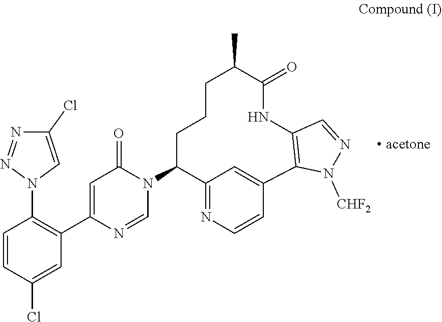
comprising the steps of:
(a) reacting Compound A having the structure: 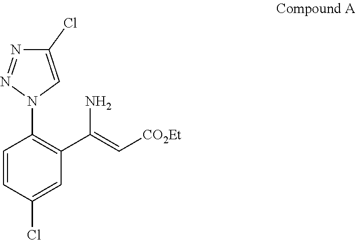 [SMILES: CCOC(=O)/C=C(\N)c1cc(Cl)ccc1-n1cc(Cl)nn1]
with N,N-dimethylformamide dimethyl acetal in a suitable solvent to yield a mixture containing methanol as a by-product;
(b) to the mixture of step (a) adding Compound C having the structure: 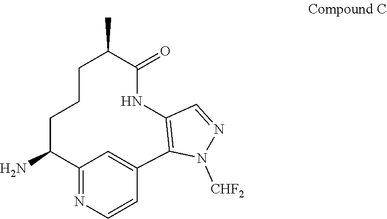 [SMILES: C[C@@H]1CCC[C@H](N)c2cc(ccn2)-c2c(cnn2C(F)F)NC1=O]
to yield the crystalline solvate form of Compound (I):

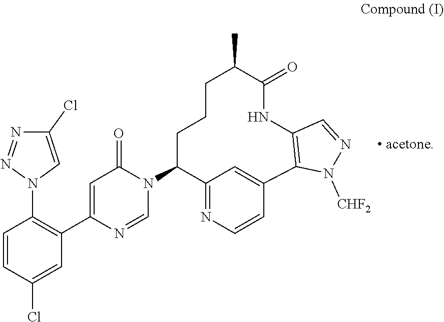

2. The process of claim 1, wherein the methanol is removed before step (b).

3. The process of claim 2, wherein acetic acid is added after the removal of methanol from the mixture of step (a).

4. The process of claim 1, wherein triethylamine is added after the addition of Compound C.

5. The process of claim 1, wherein Compound (I) is crystallized in a mixture of methanol and water followed by a rinse with aqueous acetone to yield the crystalline acetone solvate form of Compound (I).

6. The process of claim 1, wherein Compound A having the structure: 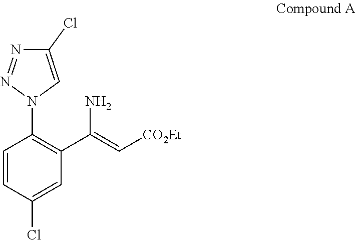 [SMILES: CCOC(=O)/C=C(\N)c1cc(Cl)ccc1-n1cc(Cl)nn1]
is prepared by a process comprising:
(a) reacting Compound 1 having the structure: 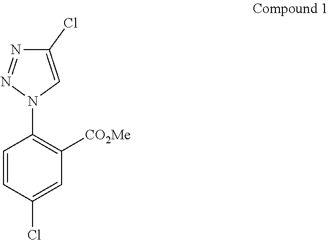 [SMILES: COC(=O)c1cc(Cl)ccc1-n1cc(Cl)nn1]
with ammonia (NH₃) in a suitable solvent to afford Compound 2 having the structure: 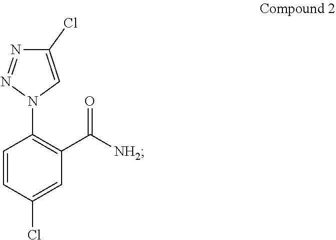 [SMILES: NC(=O)c1cc(Cl)ccc1-n1cc(Cl)nn1]
(b) reacting Compound 2 with a dehydrating agent to afford Compound 3 having the structure: 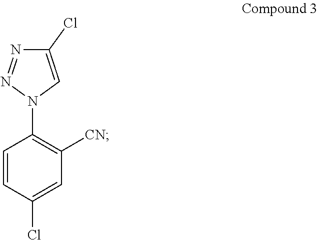 [SMILES: N#Cc1cc(Cl)ccc1-n1cc(Cl)nn1]
(c) reacting Compound 3 with potassium ethyl malonate, a base, and a Lewis Acid to afford Compound A:

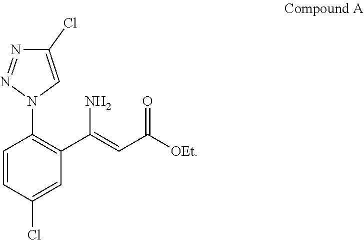 [SMILES: CCOC(=O)/C=C(\N)c1cc(Cl)ccc1-n1cc(Cl)nn1]

7. The process of claim 6, wherein calcium chloride is added as a catalyst in step (a).

8. The process of claim 6, wherein the dehydrating agent of step (b) is selected from the group consisting of phosphoroxychloride (POCl₃), (COCl)₂, PCl₅, SOCl₂ PCl₃, and dimethylchloroformiminium chloride (ClCH═N(CH₃)₂Cl.

9. The process of claim 6, wherein the Lewis Acid of step (c) is selected from the group consisting of zinc chloride (ZnCl₂), aluminum trichloride (AlCl₃), and boron trifluoride (BF₃).

10. The process of claim 6, wherein the base of step (c) is selected from the group consisting of triethylamine, N,N-diisopropylethylamine (DIPEA), 1,8-diazabicyclo[5.4.0]undec-7-ene (DBU), 1,5-diazabicyclo[4.3.0]non-5-ene (DBN), 1,4-diazabicyclo[2.2.2]octane (DABCO), tetramethylethylenediamine (TMEDA), and N,N,N′,N″,N″-pentamethyldiethylenetriamine (PMDTA).

11. The process of claim 1, wherein Compound C having the structure: 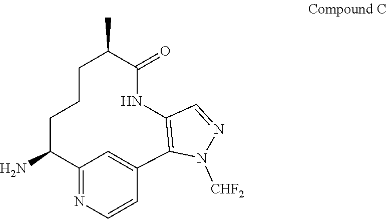 [SMILES: C[C@@H]1CCC[C@H](N)c2cc(ccn2)-c2c(cnn2C(F)F)NC1=O]
is prepared by a process comprising:
(a) dissolving the hydrochloric acid salt of Compound 15 having the structure: 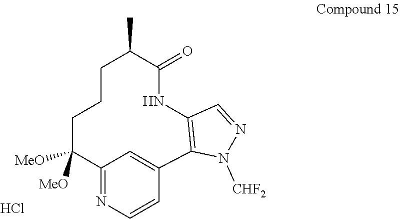
in a suitable solvent,
(b) then adding transaminase ATA-486 and an enzymatic catalyst to afford Compound C:

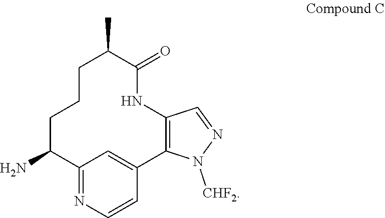 [SMILES: C[C@@H]1CCC[C@H](N)c2cc(ccn2)-c2c(cnn2C(F)F)NC1=O]

12. The process of claim 11, wherein the enzymatic catalyst is pyridoxal 5′-phosphate hydrate (5-PLP).

13. The process of claim 11, wherein the hydrochloric acid salt of Compound 15 having the structure: 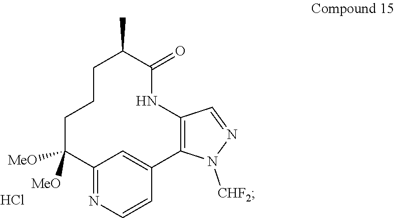
is prepared by a process comprising:
(a) reacting Compound 8 having the structure: 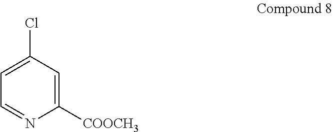 [SMILES: COC(=O)c1cc(Cl)ccn1]
with 2-methylcyclopentanone and a strong base to afford Compound 9 having the structure: 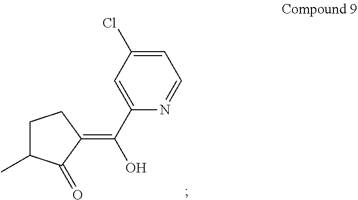 [SMILES: CC1CC/C(=C(/O)c2cc(Cl)ccn2)C1=O]
(b) reacting Compound 9 with an aqueous acid to afford Compound 10 having the structure: 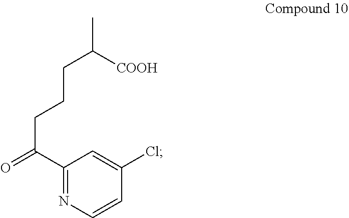 [SMILES: CC(CCCC(=O)c1cc(Cl)ccn1)C(=O)O]
(c) reacting Compound 10 with (1R,2S)-erythro-2-amino-1,2-diphenylethanol to afford the diastereomeric salt of Compound 10A: 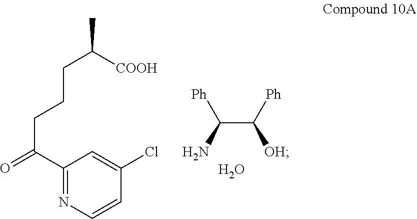
(d) dissolving the diastereomeric salt of Compound 10A with aqueous acid and a suitable organic solvent to afford Compound 10A: 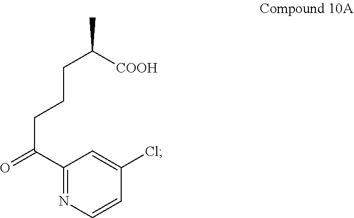 [SMILES: C[C@H](CCCC(=O)c1cc(Cl)ccn1)C(=O)O]
(e) reacting Compound 10A with trimethylsilyl chloride followed by trimethylorthoformate and then a strong base to afford Compound 11: 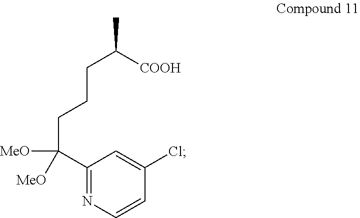 [SMILES: COC(CCC[C@@H](C)C(=O)O)(OC)c1cc(Cl)ccn1]
(f) mixing Compound 11 with dicyclohexylamine (DCHA) to afford the salt of Compound 11: 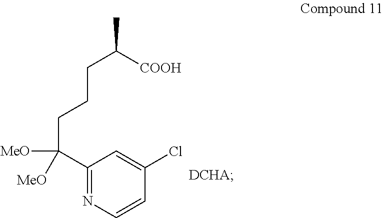 [SMILES: COC(CCC[C@@H](C)C(=O)O)(OC)c1cc(Cl)ccn1]
(g) reacting the dicyclohexylamine salt of Compound 11 with a coupling agent and the hydrochloric salt of 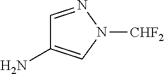
to form Compound 14 having the structure: 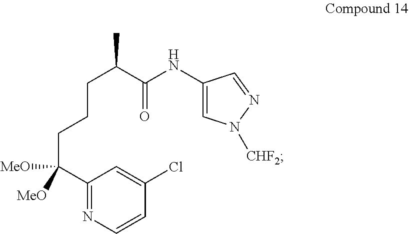
(h) Compound 14 was reacted with a metal catalyst followed by hydrochloric acid to afford the hydrochloric acid salt of Compound 15:

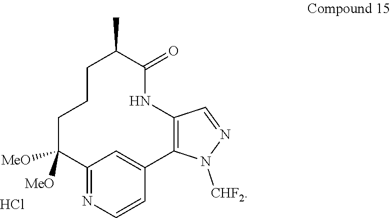

14. The process of claim 13, wherein the coupling agent of step (g) is selected from the group consisting of 1,1′-carbonyldiimidazole (CDI), dicyclohexyl carbodiimide (DCC), 1-ethyl-3-(3-dimethylaminopropyl)carbodiimide (EDCI) and 1,1′-thiocarbonyldiimidazole (TCDI).

15. The process of claim 13, wherein the metal catalyst of step (h) is selected from the group consisting of palladium, and ruthenium.

16. A crystalline form of:

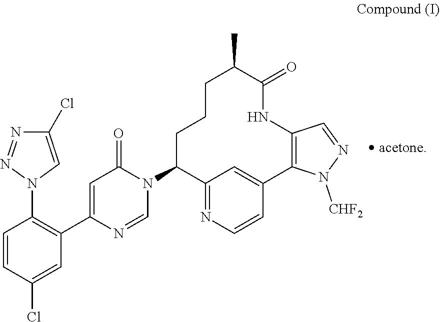 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

17. The crystalline form of claim 16 having an X-ray powder diffraction pattern comprising one, two, three or four peaks selected from peaks expressed in values of degrees 2Θ at 20.0±0.2, 21.3±0.2, 21.6±0.2, and 23.9±0.2.

18. The crystalline form of Compound (I) of claim 16, which exhibits a Fourier transform infrared spectrum having characteristic peaks expressed in units of reciprocal wave numbers (cm⁻¹) at values of about 1709, about 1676, about 1532, about 1485, about 1457, about 1441, about 1432, about 1370, about 1291, about 1219, about 1189, about 1135, about 1119, about 1068, about 1039, about 994, about 942, about 883, about 827, about 801, and about 696.

19. A process for preparing a compound represented by: 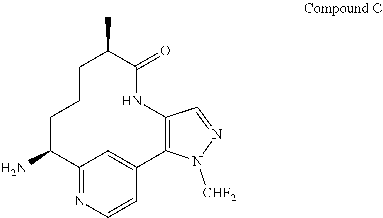 [SMILES: C[C@@H]1CCC[C@H](N)c2cc(ccn2)-c2c(cnn2C(F)F)NC1=O]
comprising:
(a) reacting Compound 17 having the structure: 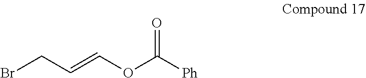 [SMILES: O=C(O/C=C/CBr)c1ccccc1]
with Compound 18 having the structure: 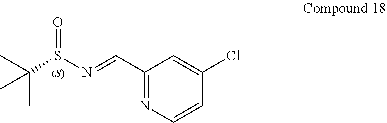
(b) subsequently adding a non-nucleophilic base followed by trimethylsilane chloride to afford the bis-hydrochloride salt of Compound 20 having the structure: 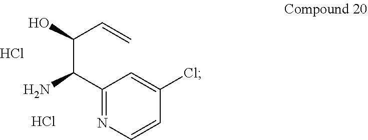
(c) reacting Compound 20 with a carbamate protecting group (PG) agent followed by propionic anhydride then a non-nucleophilic base to afford Compound 21 having the structure: 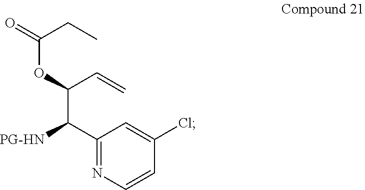
(d) reacting Compound 21 with a non-nucleophilic base to afford Compound 22 have the structure: 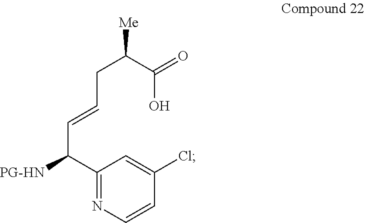
(e) reacting Compound 22 with a metal hydrogenation catalyst to afford Compound 23 having the structure: 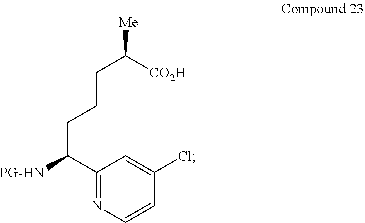
(f) reacting Compound 23 with 1-(difluoromethyl)-4-nitro-1H-pyrazole (Compound 12) and a metal catalyst to afford Compound 24 having the structure: 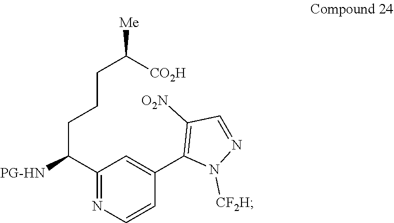
(g) reacting Compound 24 with a metal hydrogenation catalyst to afford Compound 25 having the structure: 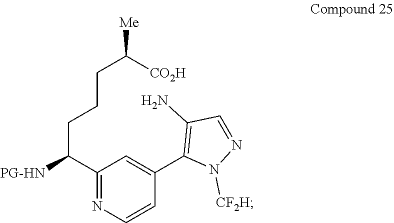
(h) reacting Compound 25 with a coupling agent to afford Compound 26 having the structure: 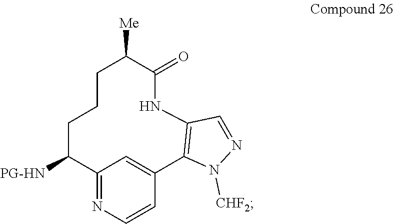
(i) reacting Compound 26 with an acid to afford Compound C:

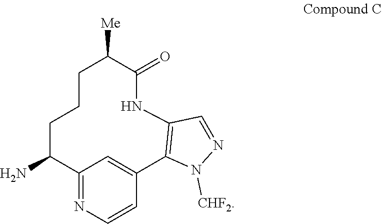 [SMILES: C[C@@H]1CCC[C@H](N)c2cc(ccn2)-c2c(cnn2C(F)F)NC1=O]

20. The process of claim 19, wherein the non-nucleophilic base is selected from the group consisting of 1,8-diazabicyclo[5.4.0]undec-7-ene (DBU), lithium bis(trimethylsilyl)amide (LiHMDS), potassium bis(trimethylsilyl)amide (KHMDS), 1,5-diazabicyclo[4.3.0]non-5-ene (DBN), and N,N-Diisopropylethylamine (Hünig's base).

21. The process of claim 19, wherein the carbamate protecting group agent of step (c) is selected from the group consisting of di-tert-butyl dicarbonate (Boc₂), carboxybenzyl chloride (Cbz-Cl), and methyl carbamate chloride (CH₃CO₂Cl).

22. The process of claim 19, wherein the metal hydrogenation catalyst is selected from carbon-supported ruthenium, Crabtree's catalyst, and carbon-supported palladium.

23. A process for the preparation of Compound 11 having the structure: 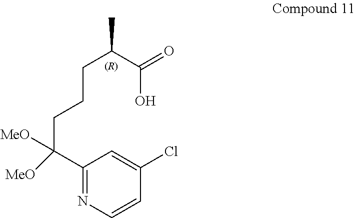
comprising the steps of:
(a) reacting Compound 27 having the structure: 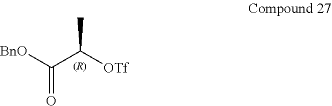
with (3-(1,3-dioxolan-2-yl)propyl)magnesium chloride to afford Compound 28 having the structure: 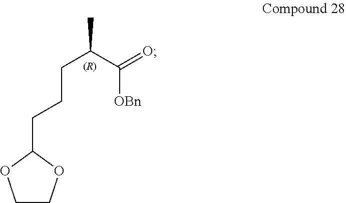
(b) reacting Compound 28 with an oxidizing agent to afford Compound 29 having the structure: 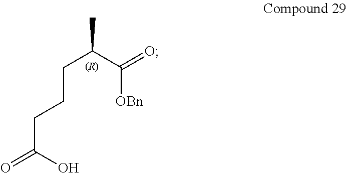
(c) reacting Compound 29 with N,O-dimethylhydroxylamine and a coupling agent to afford Compound 30 having the structure: 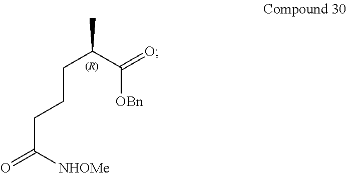
(d) reacting Compound 30 with 4-chloropyridin-2-yl magnesium bromide to afford Compound 31 having the structure: 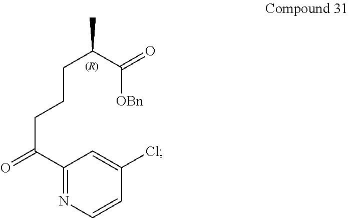
(e) subsequently adding a strong acid/alcoholic solution to afford Compound 32:

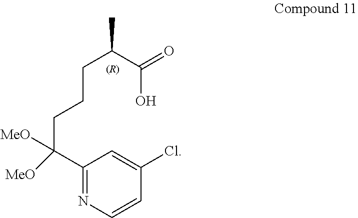

24. A process for the preparation of Compound 40 having the structure of: 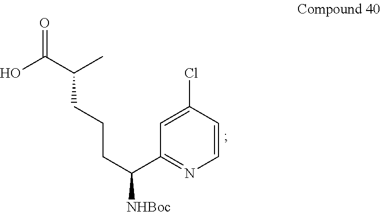 [SMILES: C[C@H](CCC[C@H](NC(=O)OC(C)(C)C)c1cc(Cl)ccn1)C(=O)O]
comprising the steps of:
(a) reacting Compound 34 having the structure: 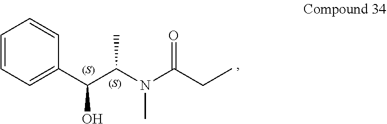
with 2-(3-bromopropyl)-1,3-dioxolane to afford Compound 35 having the structure: 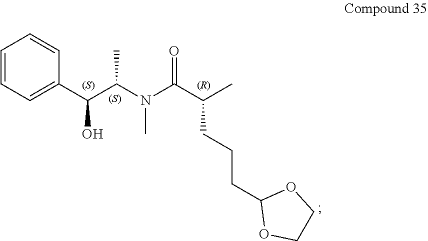
(b) reacting Compound 35 with strong base in a suitable solvent to afford Compound 36 having the structure: 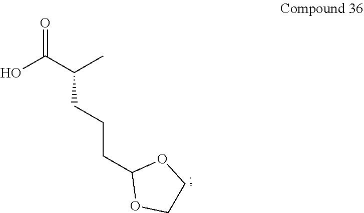
(c) reacting Compound 36 with benzyl alcohol and a coupling agent to afford Compound 37 having the structure: 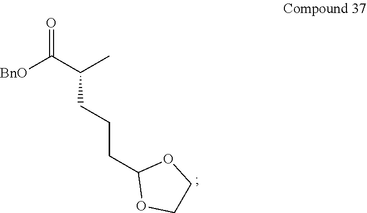 [SMILES: C[C@H](CCCC1OCCO1)C(=O)OCc1ccccc1]
(d) reacting a strong acid with Compound 37 followed by the addition of a chiral auxiliary with a metal catalyst to afford Compound 38 having the structure: 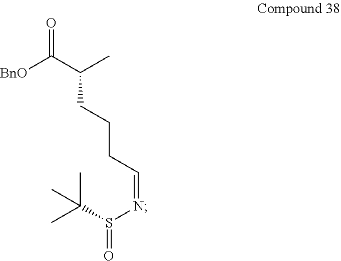 [SMILES: C[C@H](CCC/C=N\[S@](=O)C(C)(C)C)C(=O)OCc1ccccc1]
(e) subsequently reacting Compound 38 with (4-chloropyridin-2-yl)magnesium bromide to afford Compound 39 having the structure: 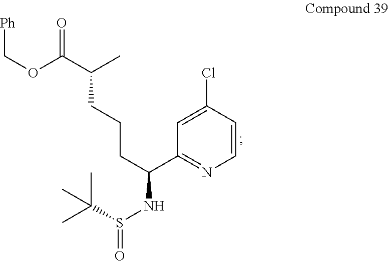 [SMILES: C[C@H](CCC[C@H](N[S@](=O)C(C)(C)C)c1cc(Cl)ccn1)C(=O)OCc1ccccc1]
(f) subsequently hydrolyzing the ester followed by the addition of a protecting group to afford Compound 40:

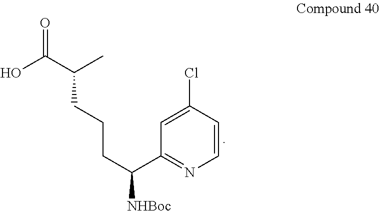 [SMILES: C[C@H](CCC[C@H](NC(=O)OC(C)(C)C)c1cc(Cl)ccn1)C(=O)O]

25. The process of claim 24, wherein the coupling agent of step (c) is selected from the group consisting of 1,1′-carbonyldiimidazole (CDI), dicyclohexyl carbodiimide (DCC), 1-ethyl-3-(3-dimethylaminopropyl)carbodiimide (EDCI) and 1,1′-thiocarbonyldiimidazole (TCDI).

26. A process for the enantiomeric enrichment of 6-(4-chloropyridin-2-yl)-2-methyl-6-oxohexanoic acid, which comprises fractional crystallization of 6-(4-chloropyridin-2-yl)-2-methyl-6-oxohexanoic acid as its salt with non-racemic 2-amino-1,2-diphenylethanol from a solution or suspension of a mixture of the enantiomers of 6-(4-chloropyridin-2-yl)-2-methyl-6-oxohexanoic acid in a solvent.

27. The process according claim 26, which comprises fractional crystallization of 6-(4-chloropyridin-2-yl)-2-methyl-6-oxohexanoic acid with 2-amino-1,2-diphenylethanol to obtain an diastereomerically enriched salt of 6-(4-chloropyridin-2-yl)-2-methyl-6-oxohexanoic acid with 2-amino-1,2-diphenylethanol and a mother liquor containing non-racemic 6-(4-chloropyridin-2-yl)-2-methyl-6-oxohexanoic acid, where the process further comprises racemisation of the 6-(4-chloropyridin-2-yl)-2-methyl-6-oxohexanoic acid contained in the mother liquor via the following steps:
a) protecting the carboxylic acid of 6-(4-chloropyridin-2-yl)-2-methyl-6-oxohexanoic acid by converting it into the corresponding C₁-C₄-alkyl ester group;
b) racemizing the carboxylic acid protected derivative of 6-(4-chloropyridin-2-yl)-2-methyl-6-oxohexanoic acid obtained in step a) by treatment with a strong base having no or low nucleophilicity, which is preferably selected from amide bases, alkaline metal salts of tertiary alkanols and sodium hydride;
c) deprotecting the racemized compound obtained in step b) to generate 6-(4-chloropyridin-2-yl)-2-methyl-6-oxohexanoic acid in the form of its racemate.

28. A process for preparing Compound 1 having the structure: 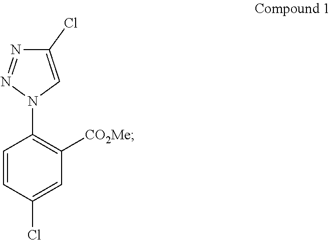 [SMILES: COC(=O)c1cc(Cl)ccc1-n1cc(Cl)nn1]
comprising the steps of
(a) reacting Compound 4 having the structure: 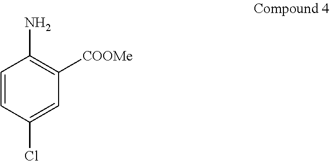 [SMILES: COC(=O)c1cc(Cl)ccc1N]
(i) with sodium nitrite and an acid in a suitable solvent to form a first mixture;
(ii) then adding the first mixture to an aqueous solution comprising sodium azide and a weak base to form a second mixture;
(iii) reacting the second mixture with trimethylsilylacetylene and copper (I) iodide (CuI) and a ligand to afford Compound 7 having the structure: 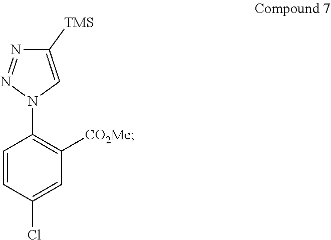 [SMILES: COC(=O)c1cc(Cl)ccc1-n1cc([Si](C)(C)C)nn1]
and
(b) chlorinating Compound 7 with a chlorinating agent to afford Compound 1.

29. The process of claim 28, wherein the acid of step (a)(i) is selected from the group consisting of methane sulfonic acid, HBF₄, TsOH, H₂SO₄ and HCl; and the weak base of step (a)(ii) is selected from the group consisting of sodium bicarbonate (NaHCO₃), potassium carbonate, pyridine, 2,6-lutidine, methylamine, triethylamine, and DMF.

30. The process of claim 28, wherein the ligand of step (a) (iii) is selected from the group consisting of tetramethylethlenediamine (TMEDA), NEt₂, DIPEA, TMEDTA, triethylamine, N,N-diisopropylethylamine, and N,N,N′,N″,N″-pentamethyl-diethylenetriamine.

31. The process of claim 28, wherein the chlorinating agent of step (b) is selected from the group consisting of 1,3-dichloro-5,5-dimethylhydantoin, NCS, NaClO, and trichloroisocyanuric acid.

32. A process for preparing Compound 1 having the structure: 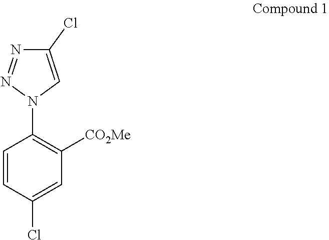 [SMILES: COC(=O)c1cc(Cl)ccc1-n1cc(Cl)nn1]
comprising the steps of:
(a) reacting Compound 4 having the structure: 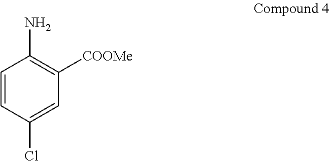 [SMILES: COC(=O)c1cc(Cl)ccc1N]
(i) with an acid and sodium nitrite in a suitable solvent;
(ii) reacting with an aqueous mixture of sodium azide and an aqueous weak base to form Compound 6 having the structure: 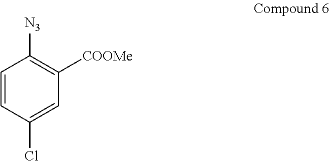 [SMILES: COC(=O)c1cc(Cl)ccc1N=[N+]=[N-]]
(b) subsequently coupling Compound 6 to chloroacetylene in the presence of a metal catalyst and a ligand to afford Compound 1.

33. The process of claim 32 wherein the acid of step (a)(i) is selected from the group consisting of methane sulfonic acid, HBF₄, TsOH, H₂SO₄ and HCl; and the weak base of step (a)(ii) is selected from the group consisting of sodium bicarbonate (NaHCO₃), potassium carbonate, pyridine, 2,6-lutidine, methylamine, triethylamine, and DMF.

34. The process of claim 32 wherein in step (b) the metal catalyst is selected from the group consisting of copper (I) iodide, CuBr, CuCl, Cu₂O, CuSO₄, CuSO₄ (5H₂O), Cu(OAc)₂, Cu(acac)₂, and CuCl₂ and the ligand is selected from the group consisting of tetramethylethlenediamine (TMEDA), NEt₂, DIPEA, TMEDTA, triethylamine, N,N-diisopropylethylamine, and N,N,N′,N″,N″-pentamethyldiethylenetriamine.
